(19) United States
(12) Patent Application Publication
(10) Pub. No.: US 2008/0229436 A1
(43) Pub. Date: Sep. 18, 2008

(54) METHODS FOR IDENTIFYING MODULATORS OF LIFESPAN AND RESISTANCE TO OXIDATIVE STRESS

(75) Inventors: Robert J.S. Reis, Little Rock, AR (US); Srinivas Ayyadevara, Little Rock, AR (US); Cagdab Tazearslan, Little Rock, AR (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignees: BOARD OF TRUSTEES OF THE UNIVERSITY OF ARKANSAS, Little Rock, AR (US); DEPARTMENT OF VETERANS AFFAIRS, Washington, DC (US)

(21) Appl. No.: 12/049,804

(22) Filed: Mar. 17, 2008

Related U.S. Application Data
(60) Provisional application No. 60/894,975, filed on Mar. 15, 2007.

(51) Int. Cl. G01N 33/50; C12Q 1/68; C12Q 1/00

(52) U.S. Cl. 800/3; 435/6; 435/4

(57) ABSTRACT

The present invention provides methods for identifying agents that increase lifespan and increase resistance to oxidative and/or electrophilic stress. Also provided are methods for identifying biomarkers of longevity or identifying pathways governing longevity in response to phosphatidylinositol 3,4,5-triphosphate signaling.

### CROSS REFERENCE TO RELATED APPLICATIONS

This application claims the priority of U.S. provisional application No. 60/894,975, filed Mar. 15, 2007, which is hereby incorporated by reference in its entirety.

### GOVERNMENTAL RIGHTS

This invention was made with government support under P01 AG20641 awarded by the National Institutes of Health. The government has certain rights in the invention.

### FIELD OF THE INVENTION

The present invention generally relates to methods for identifying agents that increase lifespan and increase resistance to an oxidative and/or electrophilic stress.

### BACKGROUND OF THE INVENTION

Aging is an enormous problem facing post-industrial society. Age is the best predictor of risk for most of the common diseases plaguing humans, including cancer and heart disease. The underlying causal mechanisms for the gradual and progressive deterioration associated with aging is poorly understood. One way toward easing this burden, and potentially preventing many age-related diseases, is by improving our understanding of the causes of the aging process at a molecular level.

Recent advances using model organisms such as mice, flies, worms, and yeast have provided some insights into the mechanisms of aging. Interestingly, this body of research indicates that many interventions that slow the rate of aging and increase lifespan are conserved between species. Caloric restriction (CR) is one such intervention. Despite the fact that CR has been known for over 70 years to extend lifespan, and can slow aging in virtually every biological system examined, the molecular basis of the lifespan extension afforded by CR remains unclear. Many genetic manipulations are also known to extend lifespan in model organisms. For example, mutations in the insulin/IGF-1 (insulin-like growth factor-1) signaling (IIS) pathway extend lifespan, and the up-regulation of stress-responsive genes may influence lifespan through a mechanism akin to CR. There is a need, therefore, for simple, rapid, and sensitive screening methods to identify compounds that extend lifespan and/or slow the ageing process.

### SUMMARY OF THE INVENTION

Among the various aspects of the present invention is one aspect that provides a method for identifying an agent that increases resistance to oxidative and/or electrophilic stress. The method comprises contacting a test nematode with at least one agent, and then exposing the test nematode, a negative control nematode, and a mutant nematode to an oxidative and/or electrophilic stress. The mutant nematode is an extremely long-lived mutant nematode that has increased resistance to oxidative and/or electrophilic stress. The method further comprises comparing the stress responses of the test nematode, the negative control nematode, and the mutant nematode, wherein a change in the response of the test nematode away from the response of the negative control nematode and towards the response of the mutant nematode indicates that the agent provides protection against the oxidative and/or electrophilic stress.

Another aspect of the invention encompasses a method for identifying a biomarker of longevity or a pathway governing longevity in response to phosphatidylinositol 3,4,5-triphosphate (PIP3) signaling. The method comprises contacting a first nematode with at least one treatment that alters the levels or function of PIP3, and then exposing the first nematode and a second untreated nematode to an oxidative and/or electrophilic stress. The method further comprises comparing the stress responses of the first and the second nematodes, wherein an increase in resistance to the stress in the first nematode indicates that the treatment altered a longevity biomarker or a PIP3 signaling pathway that mediates longevity.

A further aspect of the invention provides a method for identifying a biomarker of longevity or a pathway governing longevity in response to phosphatidylinositol 3,4,5-triphosphate (PIP3) signaling. The method comprises contacting an extremely long-lived age-1 null mutant C. elegans nematode with at least one constitutively active PIP3 interacting protein, and then exposing the treated age-1 nematode and an untreated extremely long-lived age-1 null mutant nematode to an oxidative and/or electrophilic stress. The method further comprises comparing the stress responses of the treated and untreated age-1 null mutant nematodes, wherein a decrease in resistance to the stress in the treated age-1 null mutant nematode indicates that the protein mediates effects downstream of age-1 to confer the extreme longevity and increased resistance to oxidative and/or electrophilic stress of the extremely long-lived age-1 null mutant nematode.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### DESCRIPTION OF THE FIGURES

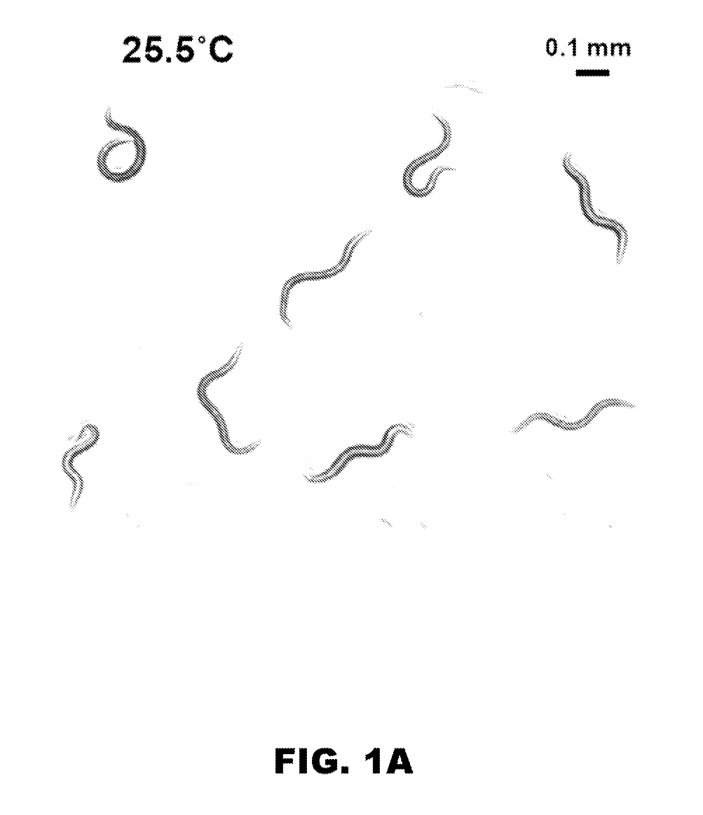

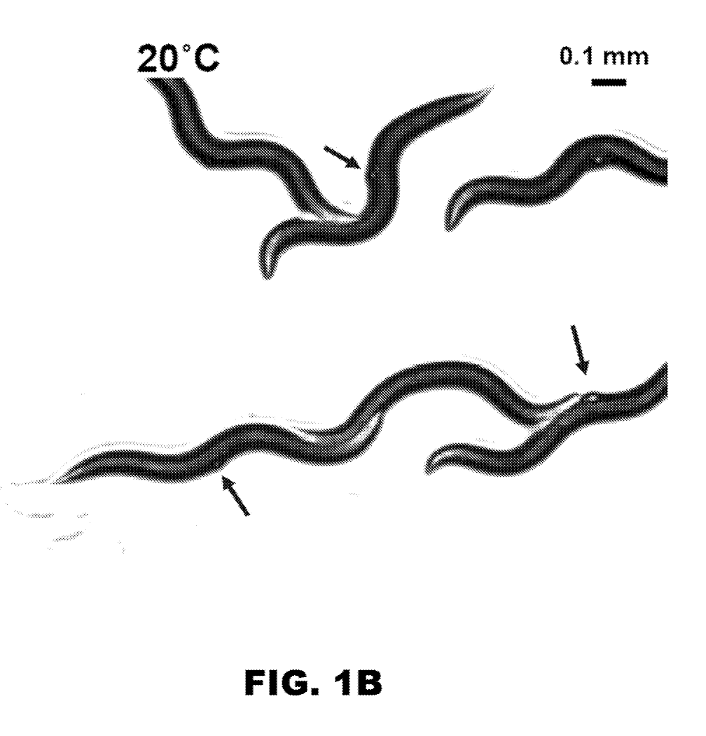

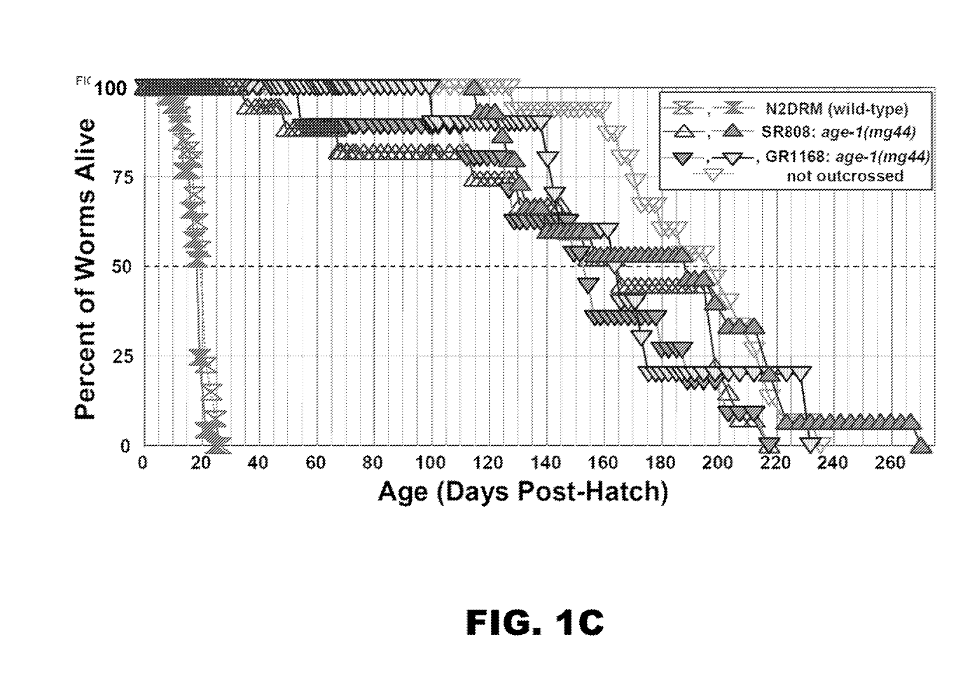

FIG. 1 illustrates that C. elegans nematodes of age-1(mg44)/age-1(mg44) genotype, born of homozygous age-1(mg44) hermaphrodites, matured at 20° C. into adults of near-normal appearance with increased lifespan. (A) Photographic image of larval arrested worms raised at 25.5° C. for 6 days after hatching. (B) Adults grown at 20° C. and imaged 9 days after hatching. (C) Survivals, on agar medium, are shown for N2DRM control worms (2 independent cultures, 50 worms each), GR1168 (second-generation age-1(mg44) homozygotes, 3×50 worms), and SR808 (derived from GR1168 by 6 generations of outcrossing to N2DRM; second-generation age-1(mg44) homozygotes, 2×50 worms). Each age-1(mg44) group differed from either N2DRM control group, by log-rank test, at P<10⁻⁹.

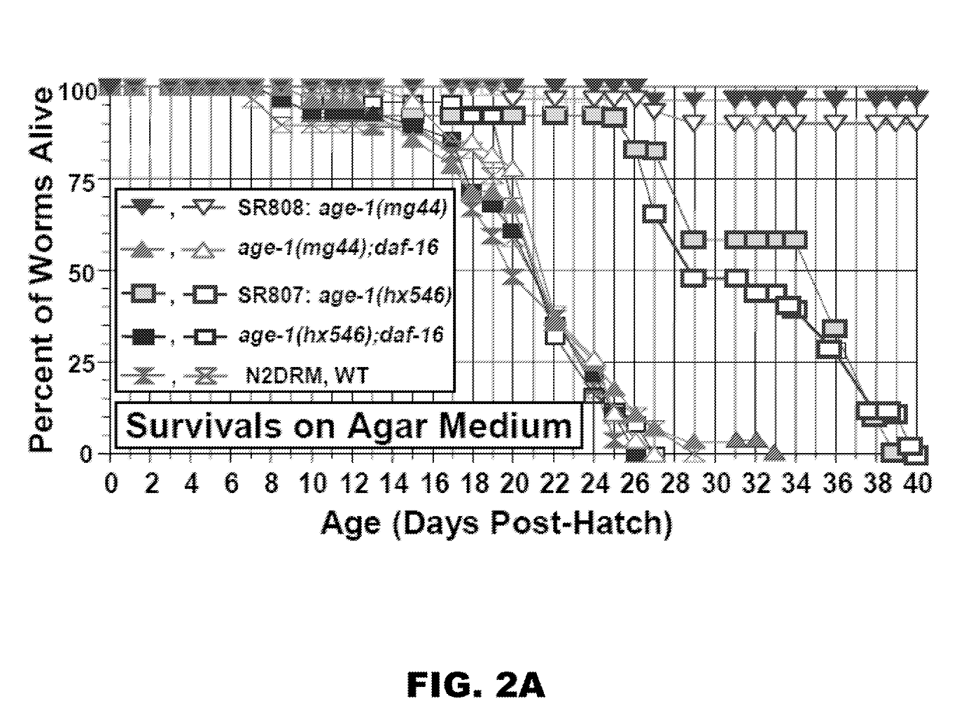

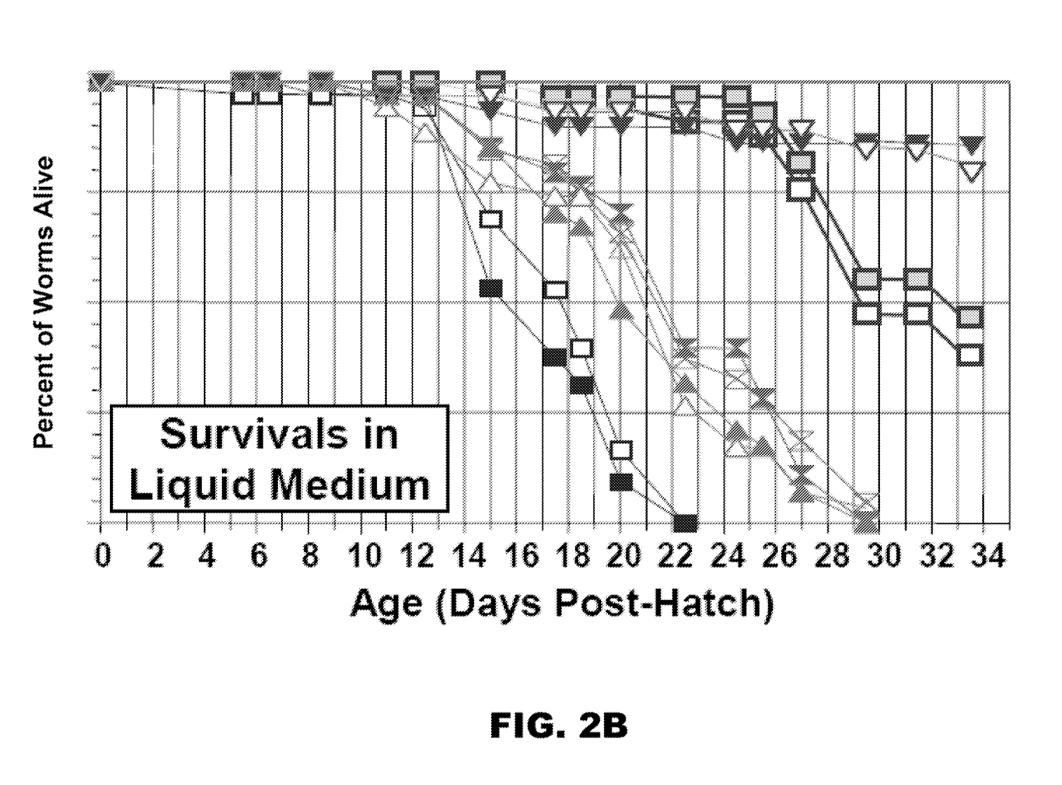

FIG. 2 illustrates that second-generation homozygous age-1(mg44) worms are longer-lived than age-1(hx546) homozygotes, and these traits are largely DAF-16/FoxO-dependent. Age-1 alleles hx546 and mg44 were crossed with daf-16(m26), all in N2DRM background; double-mutant homozygous progeny were selected and propagated. Each symbol represents 50 worms (2 plates, 25 each). (A) Lifespan survivals on solid-agar medium seeded with an E. coli OP50 lawn. (B) Lifespan survivals in liquid medium to which E. coli OP50 (10⁹/ml) were added. Statistics: (A, B) SR807 or SR808 vs. any other strain: P<10⁻⁷. (B) age-1 (hx546);daf-16 vs. other controls: P<10⁻³.

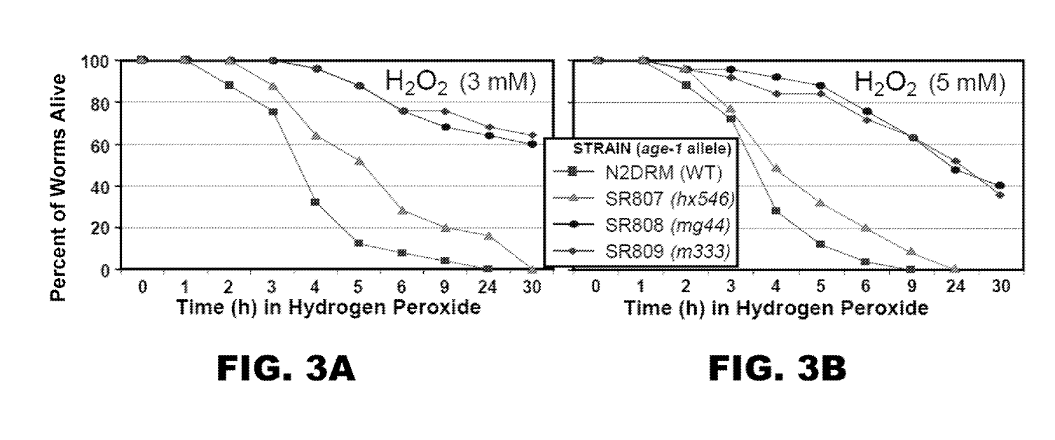

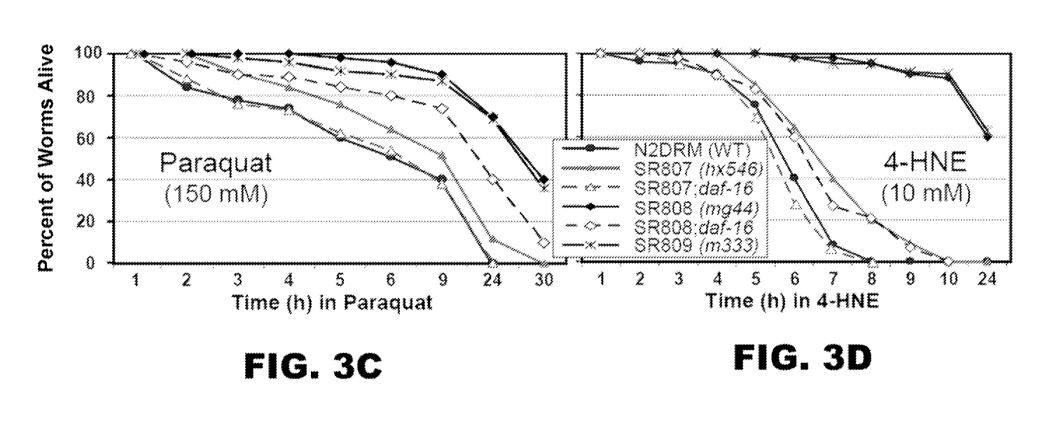

FIG. 3 illustrates that second-generation homozygous age-1(mg44) worms are more resistant to oxidative and/or electrophilic stresses than age-1(hx546) homozygotes, and these traits are largely DAF-16/FoxO-dependent. Strains are as in FIG. 2; SR809 comprises second-generation age-1(m333) homozygotes outcrossed 6 times to N2DRM. Survival cultures, maintained at 20±0.3° C., were tested at adult day 2. (A) Stress survivals in 3 mM hydrogen peroxide. (B) Survivals in 5 mM hydrogen peroxide. (C) Survivals in 150 mM paraquat. (D) Survivals in 10 mM 4-hydroxynonenal. Results of three experiments were very similar. Runts were excluded from all SR809 survivals. Statistics: (A) any age-1 strain vs. N2DRM: P<0.004. (B) SR808, SR809 vs. N2DRM: P<10⁻⁴. (C, D) SR807 vs. age-1(hx546);daf-16 or N2DRM: P<0.005; SR808, SR809 vs. age-1(hx546);daf-16 or N2DRM: P<10⁻⁹.

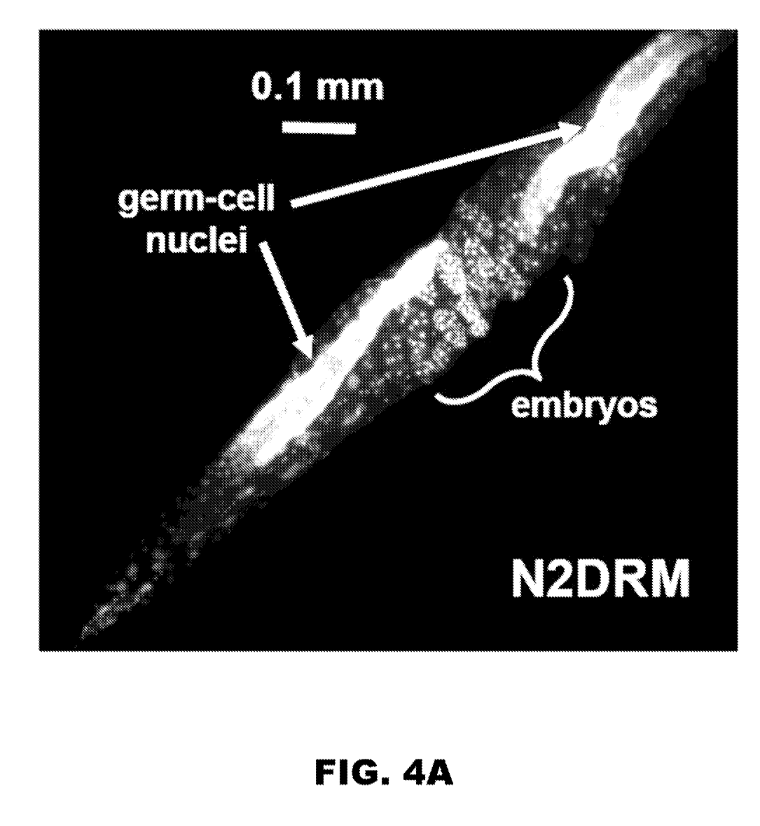

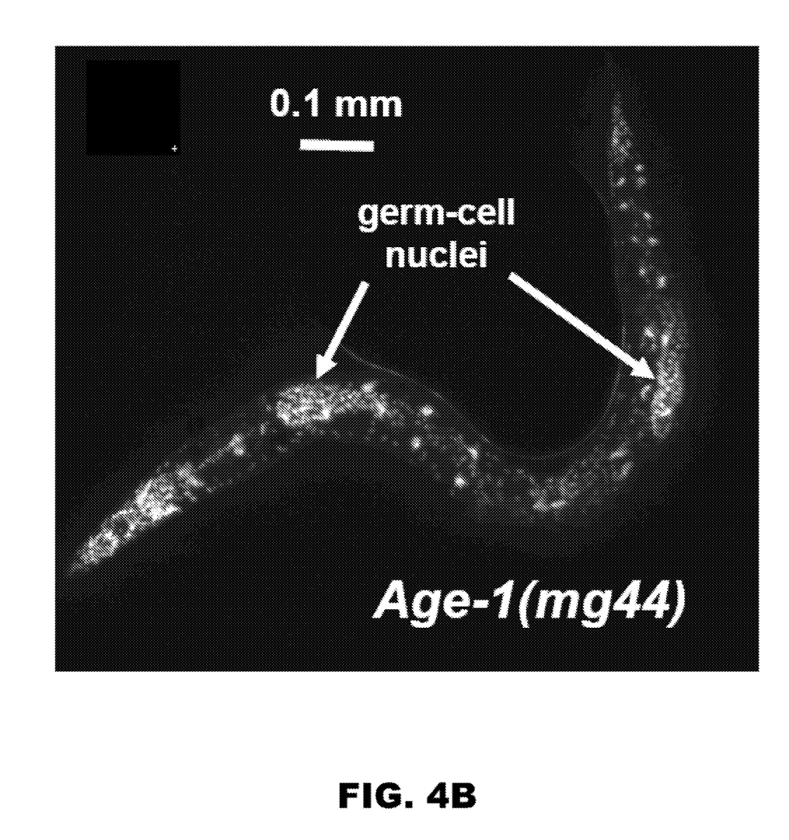

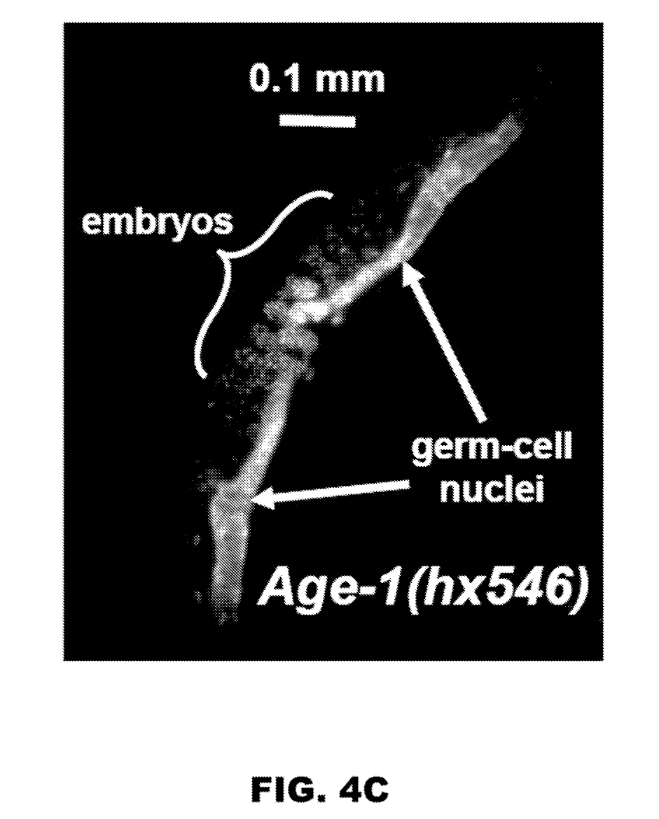

FIG. 4 illustrates that second-generation homozygous age-1(mg44) worms have reduced numbers of germ cells and form no embryos. Day-1 adult worms were stained with DAPI (4′,6-diamidino-2-phenylindole) and imaged by fluorescence microscopy (350-nm excitation, 450-nm emission) to highlight nuclear DNA. (A) N2DRM control worm. (B) Age-1(mg44) second-generation homozygote. (C) Age-1(hx546) homozygous worm.

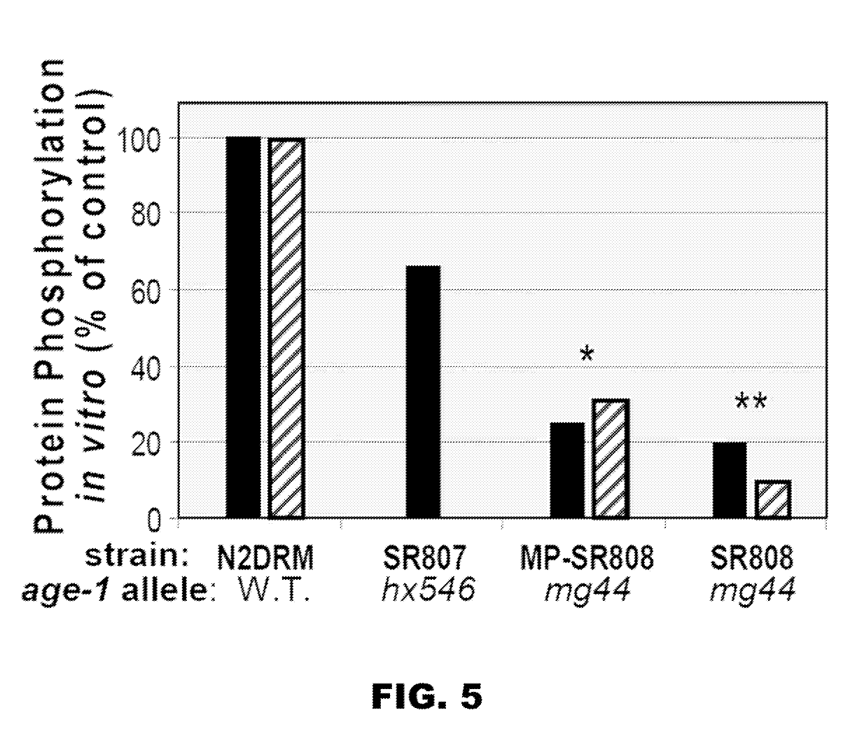

FIG. 5 illustrates that age-1 mutant worms have reduced protein phosphorylation. Protein-kinase activity for endogenous substrates, measured as in vitro incorporation of ³²P-Y-ATP into discrete high-molecular-weight products (>25 kDa) during 60 sec at 30° C., by cleared worm lysates (10,000-g supernatant) of day-5 adults. Results comprise two independent grow-ups of N2DRM (controls), SR808 [age-1(mg44)], and maternally-protected SR808 (MPSR808), and one assay for SR807 [age-1 (hx546)]. SR808 worms differ from N2DRM by single tailed t-test (corrected for multiple assays): *P<0.03, **P<0.002. SR808 worms differ from MPSR808: P<0.04; F-test for significantly differing variances between these two groups, P=0.6.

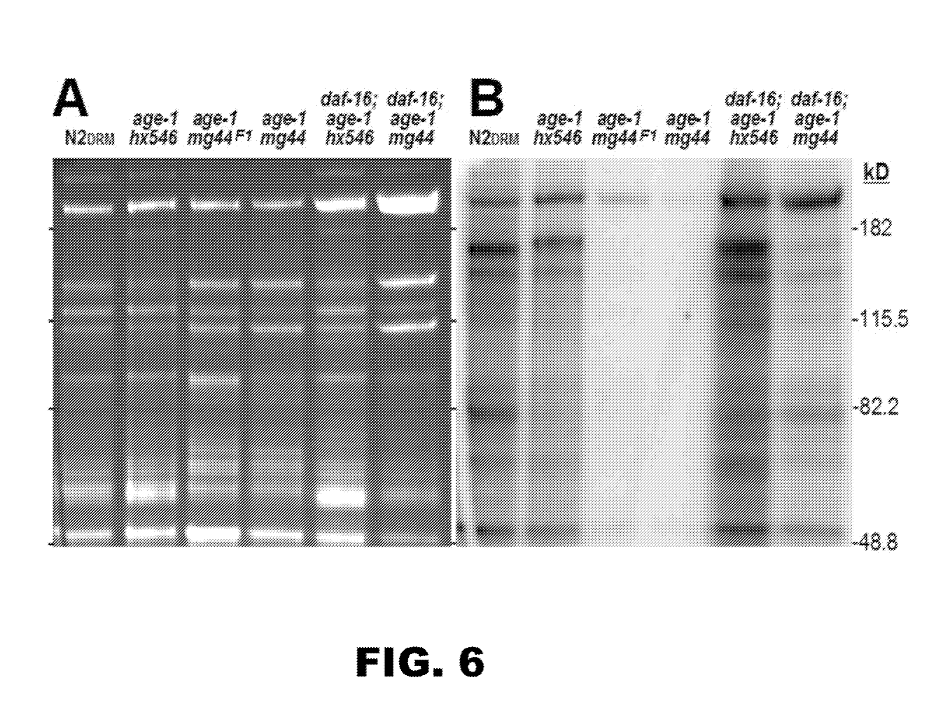

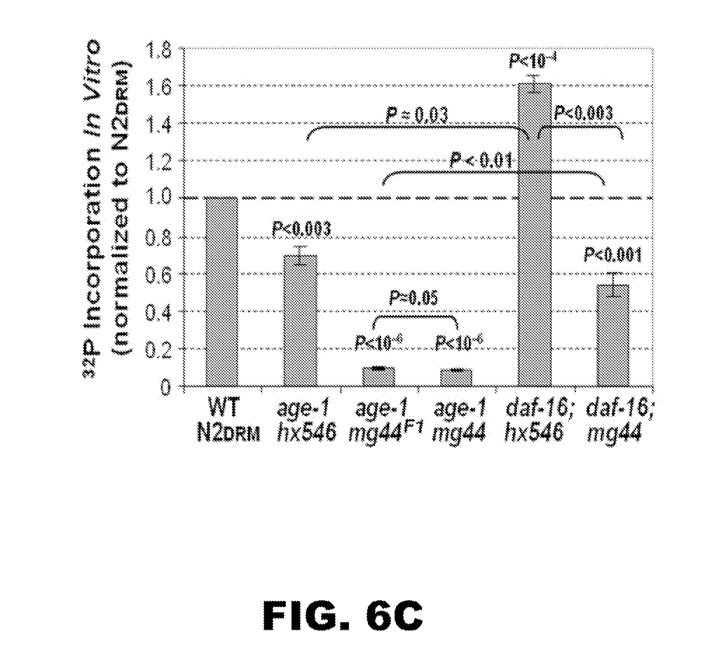

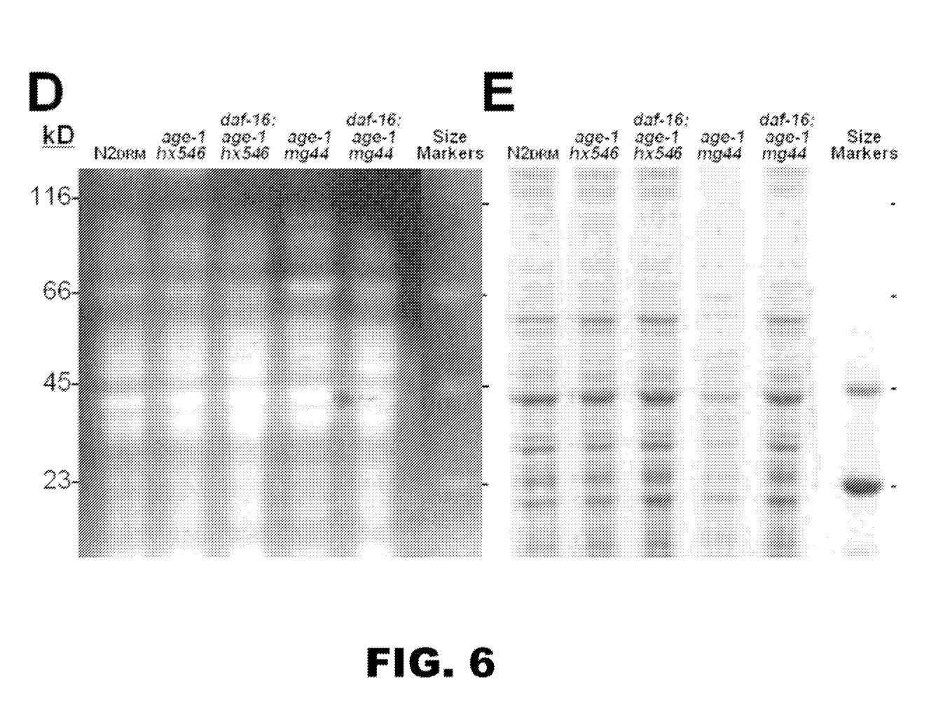

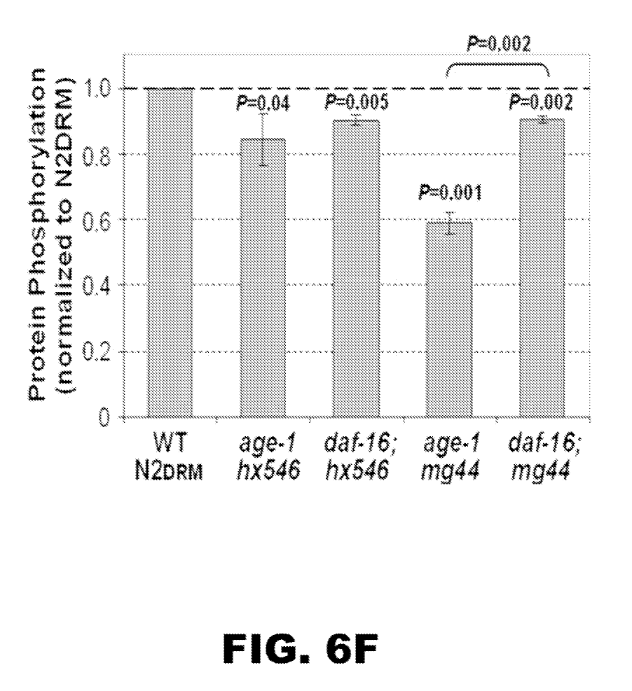

FIG. 6 illustrates that both in vitro kinase activity for endogenous substrates and total phosphoprotein levels are diminished in age-1(mg44) homozygotes. (A-C) In vitro kinase activity of day-6 adult worms (6 days after the L4/adult molt). Kinase activity of cleared, sonicated lysates was assessed as incorporation of γ-³²P-ATP per 20 μg protein sample, in 1 min at 30° C. Samples were quenched on ice and electrophoresed on 10% polyacrylamide-SDS gels. (A) Gels stained with SYPRO® Ruby to confirm constant protein loads. (B) Image of ³²P β-emissions from the gel in A, dried under vacuum. (C) Data summary from 2-3 independent expansions each of N2DRM, age-1(hx546), age-1(mg44) F1 homozygotes (labeled age-1 mg44^{F1}), age-1(mg44) F2 homozygotes (labeled age-1 mg44), and daf-16(m26) double mutants with each age-1 allele. (D-F) Phosphoproteins, visualized by Pro-Q Diamond staining after polyacrylamide gel electrophoresis. (D) Image of total protein stained with Coomassie blue. (E) Image of fluorescence after Pro-Q Diamond staining; note that only the two phosphoprotein markers were detected. (F) Summary of data from 3 independent expansions of each strain at adult day 6. Significance: (C, F), P values are shown for 2-tailed t-tests (unequal variance) comparing each age-1 strain to N2DRM (values directly over bars) or comparing two strains connected by brackets (values over brackets).

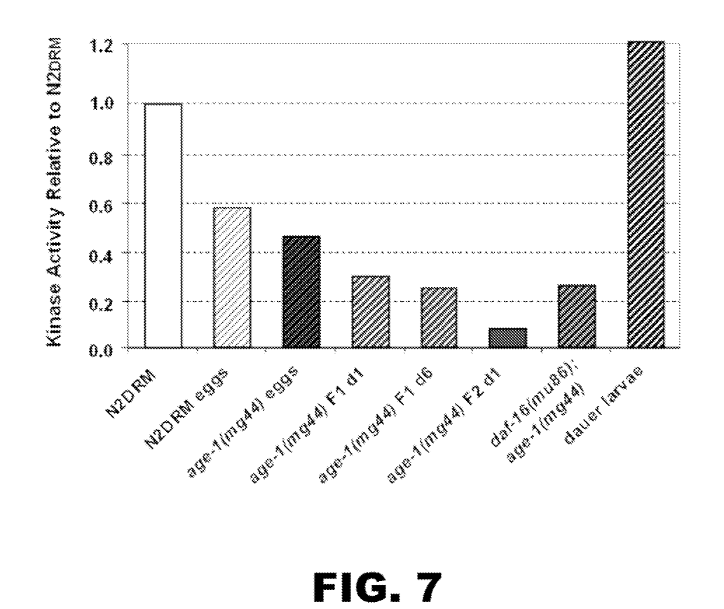

FIG. 7 shows that in vitro kinase activity for endogenous substrates is reduced in age-1(mg44) homozygotes, but not in dauer larvae. Kinase activity was assessed for N2DRM day-6 adults and eggs, age-1(mg44) F1 eggs, day-1 and day-6 adults, age-1(mg44) F2 day-1 adults, daf-16(mu86); age-1(mg44) adults, and N2DRM dauer larvae. Kinase activity of cleared, sonicated lysates was assessed as described in the legend to FIG. 6.

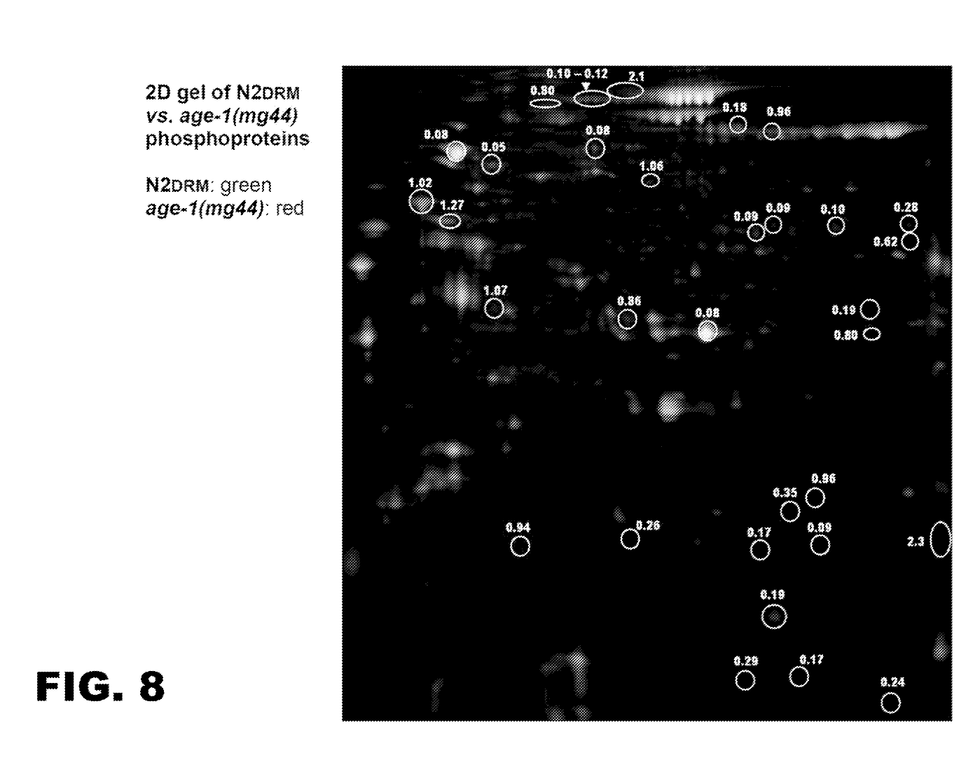

FIG. 8 presents a typical 2-dimensional polyacrylamide gel stained for phosphoproteins (enriched using phosphoaffinity columns) from age-1(mg44) F2 adult worms (red fluorescence) and from near-isogenic N2DRM controls (green fluorescence). A total of 1669 spots were identified and quantified for each fluor after co-electrophoresis. Red/green ratios equal to or greater than 2 (i.e., at least 2-fold more abundant in age-1(mg44) than in N2DRM adults) were observed for 13 spots, 30 had ratios of 1.2-2.0, 122 were essentially constant (ratios of 0.8-1.2), 305 fell between 0.5 and 0.8, and 1199 were reduced at least twofold in abundance in age-1(mg44) adults. A reduction of at least 3-fold was seen for 784 spots, and ≧5-fold for 287 spots.

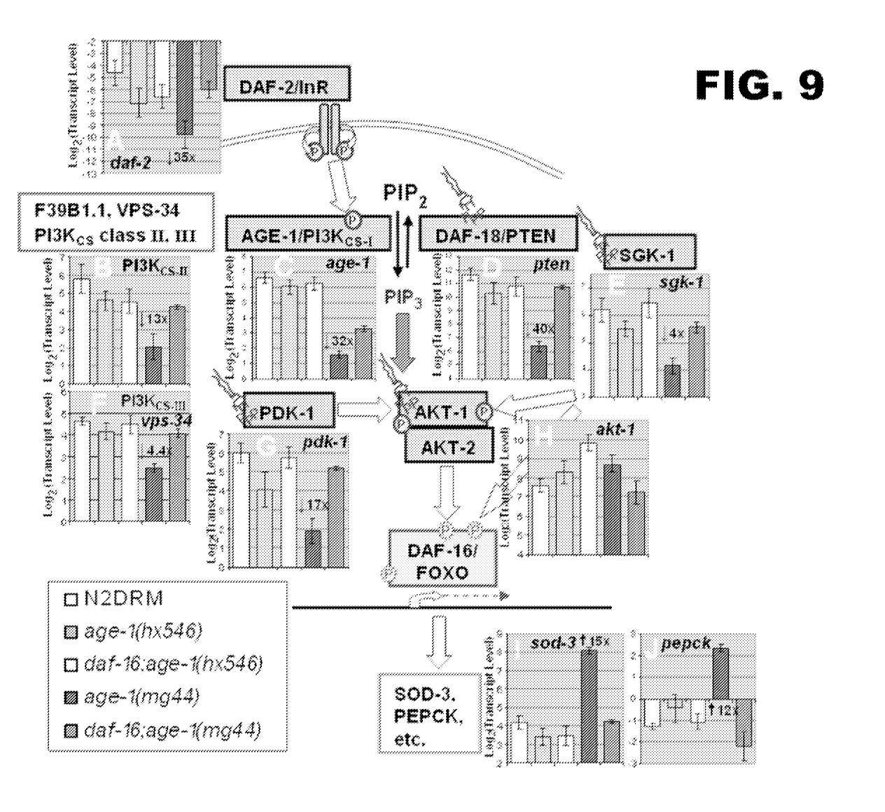

FIG. 9 depicts transcript levels of the IIS pathway genes as determined by real-time PCR for 2-4 biological replicates of each strain. Expression histograms are superimposed on a IIS pathway diagram, in which phosphorylations are indicated by yellow arrows leading to encircled P's, and phosphatidylinositol 3,4,5-triphosphate (PIP3) binding is indicated by small red diagrams. Significance: Differences, tested by a 2-tailed t test, considering only independent biological grow-ups, were significant for age-1(mg44) relative to N2DRM, or relative to its double mutant with daf-16 (indicated here in parentheses), wherein most age-1 effects are largely or entirely reverted, as follows: (A) daf-2 transcripts declined 35× (13.5×); P=0.01 (P<0.04). (B) F39B1.1 (pi3k_{cs-III}) transcripts declined 13× (5×); P<0.004 (P<0.03). (C) age-1(pi3k_{cs-I}) transcripts declined 32× (3.2×); p<10⁻⁶ (P<10⁻⁵). (D) daf-18 (pten) transcripts fell 40× (20×); P<10⁻⁴ (P<10⁻⁴). (E) sgk-1 transcripts fell 4× (3×); P<0.001 (P<0.002). (F) vps-34 (pi3k_{cs-III}) transcripts dropped 4.4× (3×); P<10⁻⁵ (P<0.0003). (G) pdk-1 transcripts declined 17× (10×); P<0.002 (P<0.004). (H) akt-1 transcripts rose 2.2× (2.8×), N.S. (N.S.). (I) sod-3 transcripts increased 15× (15×), P<10⁻⁴ (P<10⁻⁴). (J) pepck transcripts increased 12× (23×), p<10⁻⁴ (P<0.002).

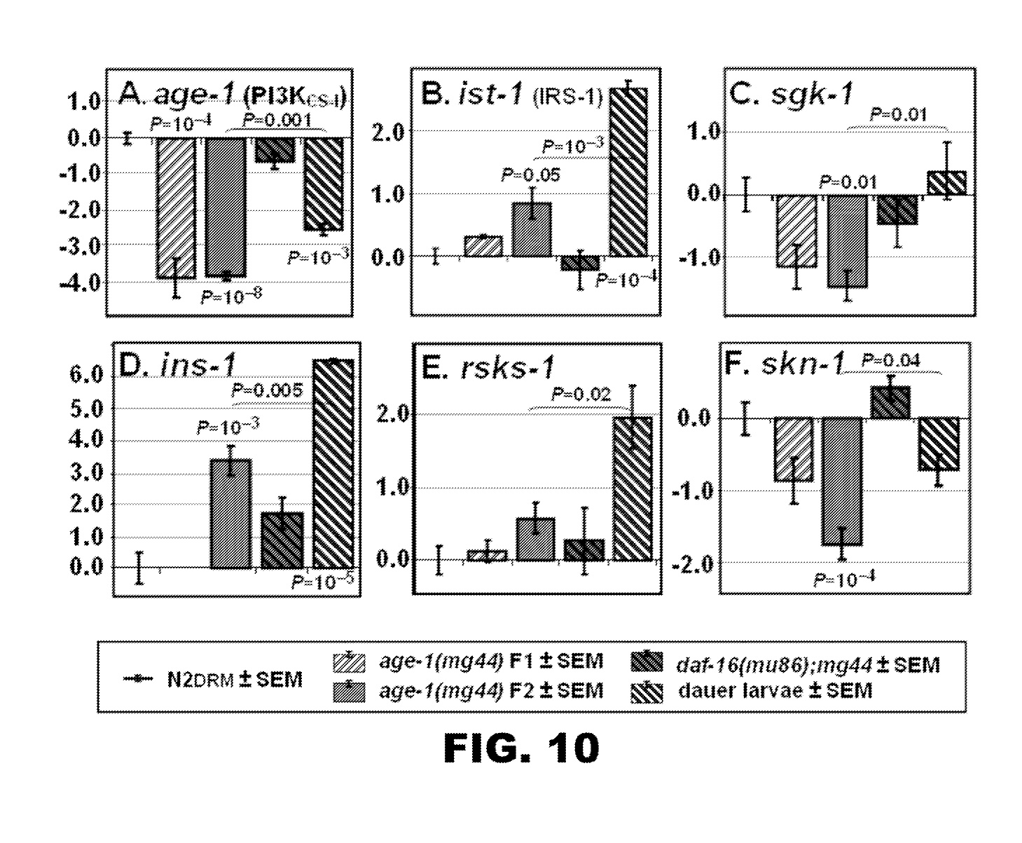

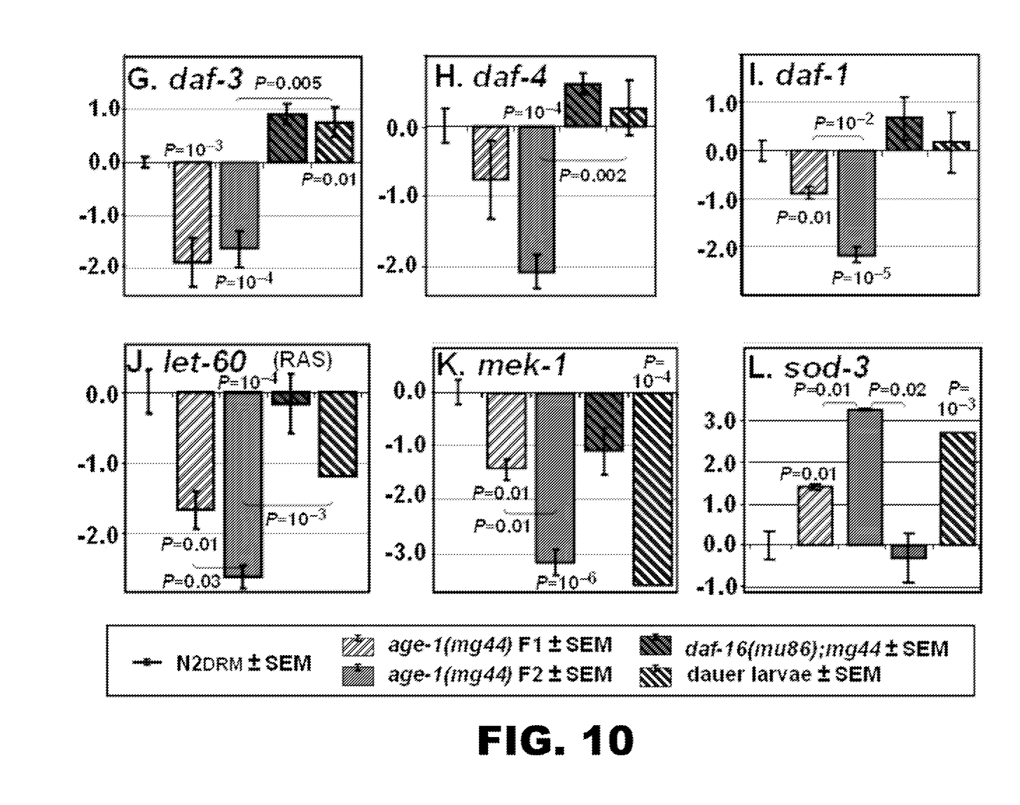

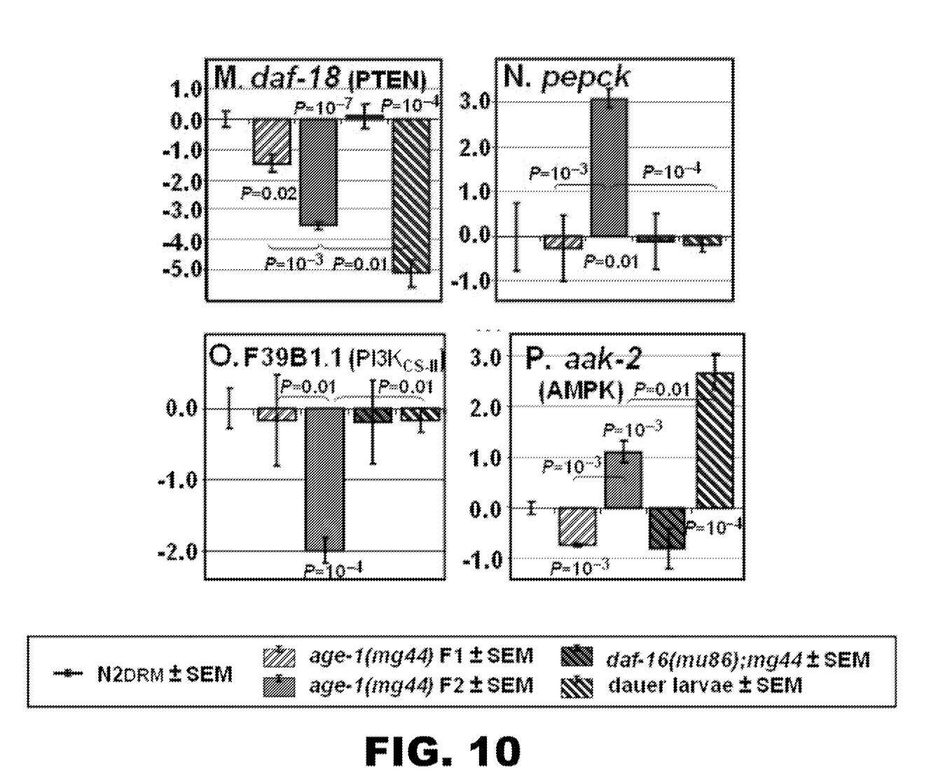

FIG. 10 illustrates that the age-1(mg44) F2 transcript profile is distinct from those of F1 adults and dauer larvae. Error bars and significances reflect variation among biological replicates, of which there were 2-14 per group (except J-L dauer samples, which were single assays). Significant differences between F2 age-1(mg44) and N2DRM adults became insignificant in double mutants with daf-16(mu86). P values directly over bars indicate significant differences vs. N2DRM; P values over brackets relate groups connected by brackets. Significance was assessed by Behrens-Fisher t-test (unequal variance), or Z values (normal-distribution deviation) for the three “singlicate” assays. (A-H) Signal transduction genes differ significantly between F2 age-1(mg44) adults and dauer larvae. Gene (encoded protein): (A) age-1 (PI3K_{CS-I}), (B) ist-1 (insulin receptor substrate 1), (C) sgk-1 (serum/glucocorticoid-stimulated kinase 1, (D) ins-1 (insulin-like peptide 1, an IIS antagonist), (E) rsks-1 (S6 ribosomal-protein kinase), (F) skn-1 (skinhead transcription factor), (G) daf-3 (co-SMAD protein), (H) daf-4 (membrane Ser/Thr kinase). (I-P) Signal transduction genes differ significantly between F2 and F1 age-1(mg44) adults. (I) daf-1 (TGF-β receptor), (J) let-60 (RAS GTPase), (K) mek-1 (MAPKK), (L) sod-3 (Mn⁺⁺ superoxide dismutase), (M) daf-18 (PTEN PIP3 phosphatase), (N) R11A5.4 (phosphenolpyruvate carboxykinase), (O) F39B1.1 (PI3KCS-II), (P) aak-2 (AMP-dependent protein kinase, catalytic subunit 2).

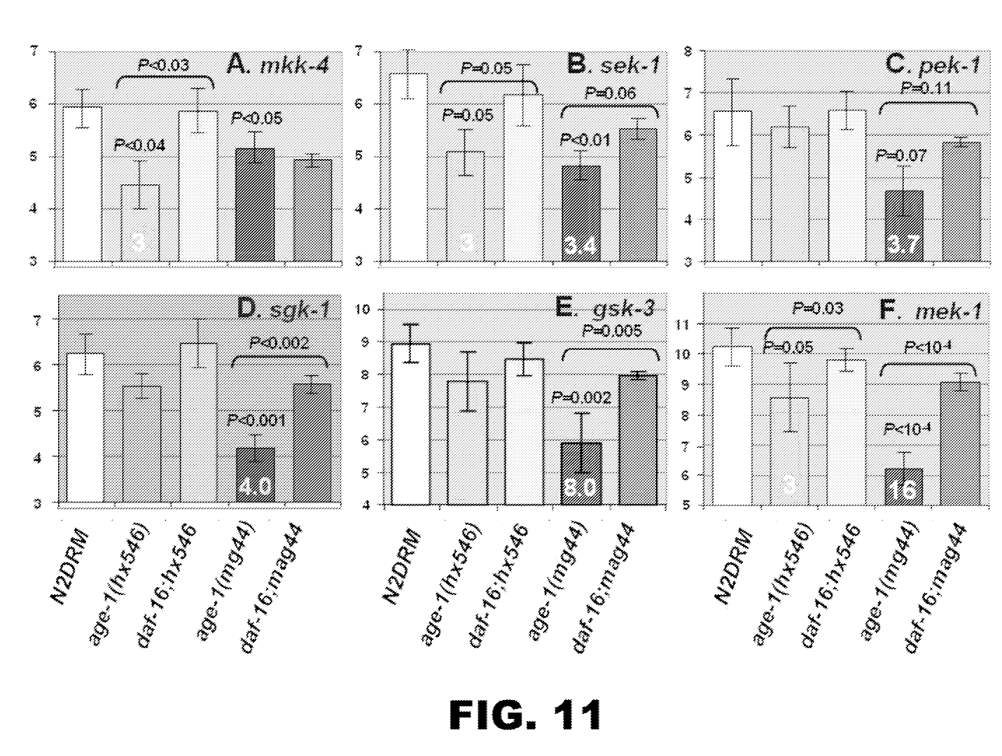

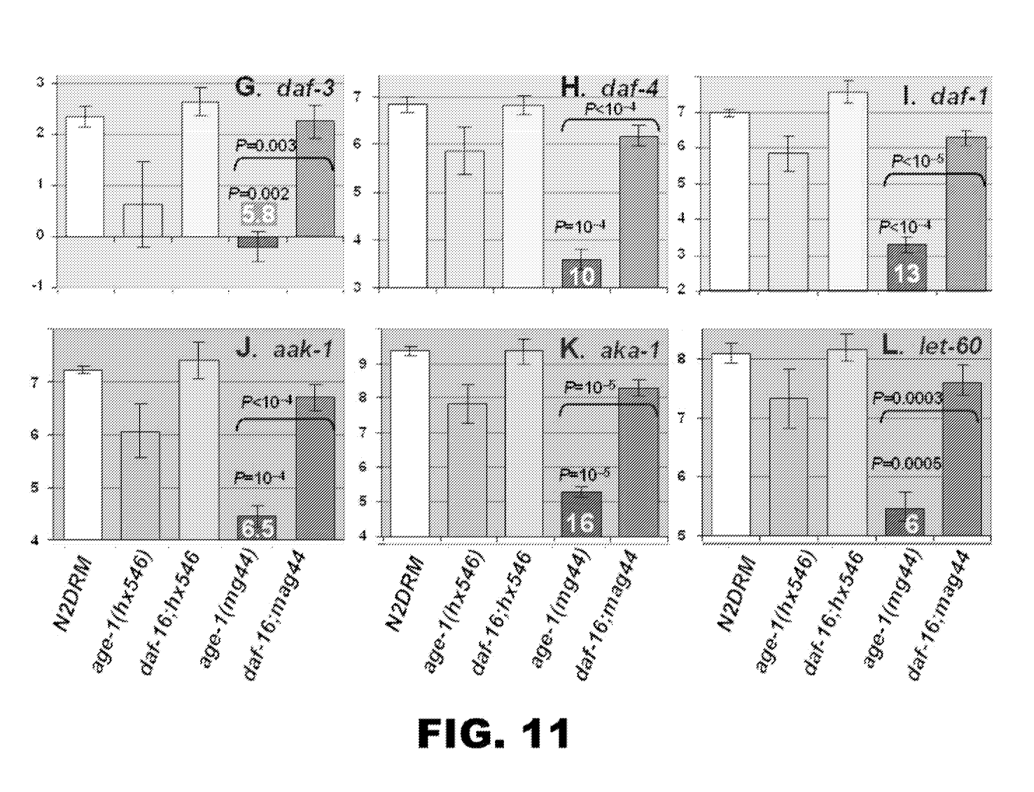

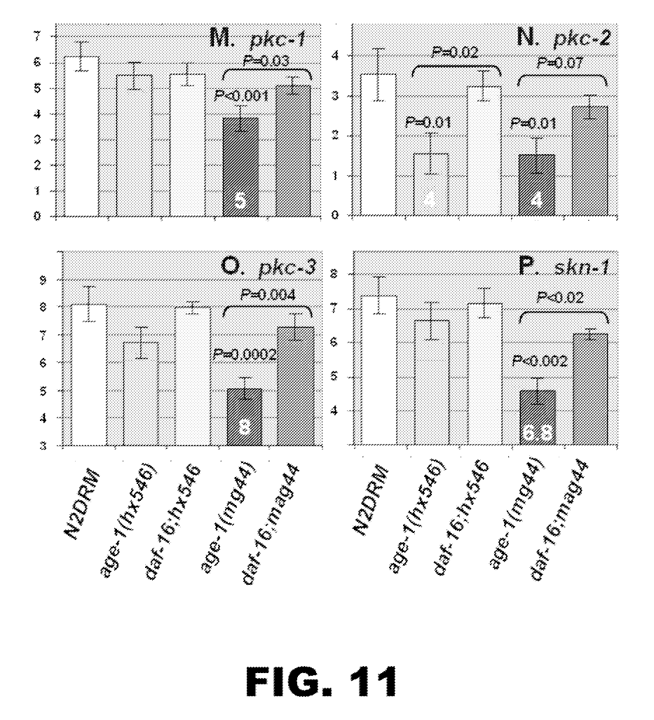

FIG. 11 depicts transcript levels for genes that may interact with IIS components. Means±SEM are shown on a log₂ scale for steady-state transcript level in wild-type N2DRM worms and age-1 mutant strains at day 6 of adulthood. Transcript levels were determined by real-time PCR from two cDNA syntheses for each of two independent biological replicates, normalized to the mean of two internal control genes that did not alter significantly among the strains. Statistics: Significances are shown for 2-tailed t-tests, regardless of whether the direction of change was predicted, comparing age-1(mg44) second-generation homozygotes to N2DRM (numbers just above age-1 error bars, without brackets), or to daf-16; age-1(mg44) double mutants (over brackets). All t-tests use an N of 2, the number of biological replicates. Genes (protein product): A, mkk-4 (neuronal mitogen-activated kinase kinase [MAPKK]); B, sek-1(a MAPKK); C, pek-1 (eIF2α kinase); D, sgk-1 (homolog of serum/glucocorticoid-inducible kinase; interacts with PDK-1 and P13K); E, gsk-3 (glycogen-synthase kinase); F, mek-1 (MKK7, a MAPKK); G, daf-3, a SMAD transcription factor; H, daf-4, Ser/Thr kinase, type-II TGF-β receptor; I, daf-1, Ser/Thr kinase, type-I TGF-β receptor; J, aak-1, AMP-activated kinase, catalytic subunit (AMPKCS); K, aka-1, A-kinase anchor protein; L, let-60, a RAS-family GTPase known to interact with 67 genes incl. clk-1, age-1, and β-catenin; M-O, pkc-1, -2, and -3 (protein kinase C paralogs); P, skn-1 (bZip transcription factor, responsive to oxidative stress).

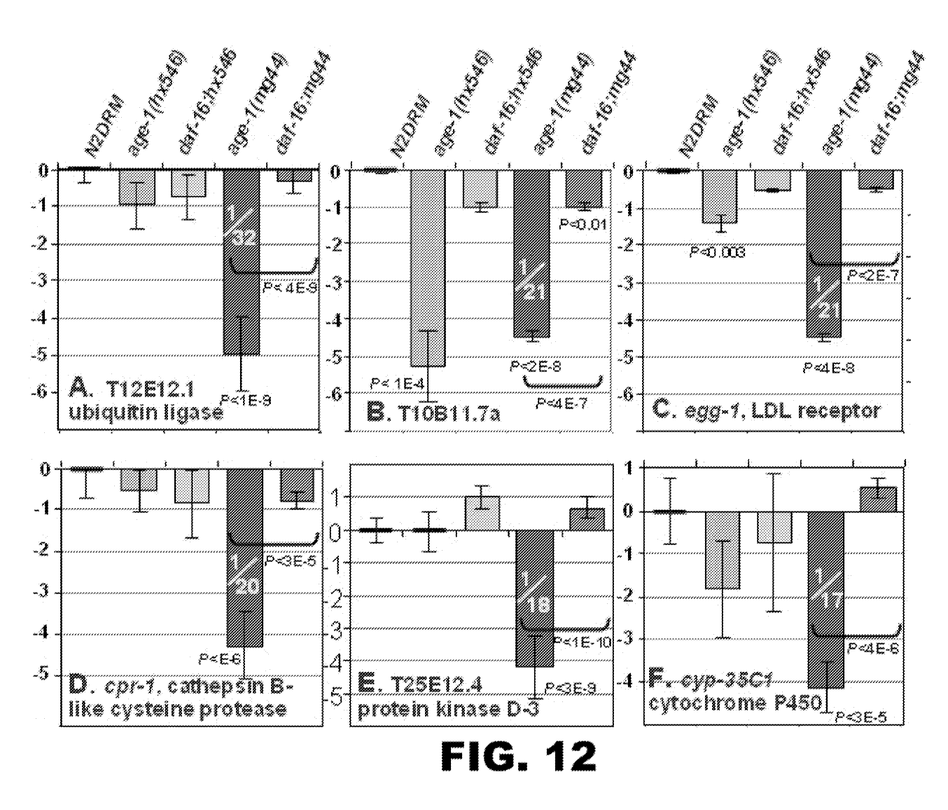

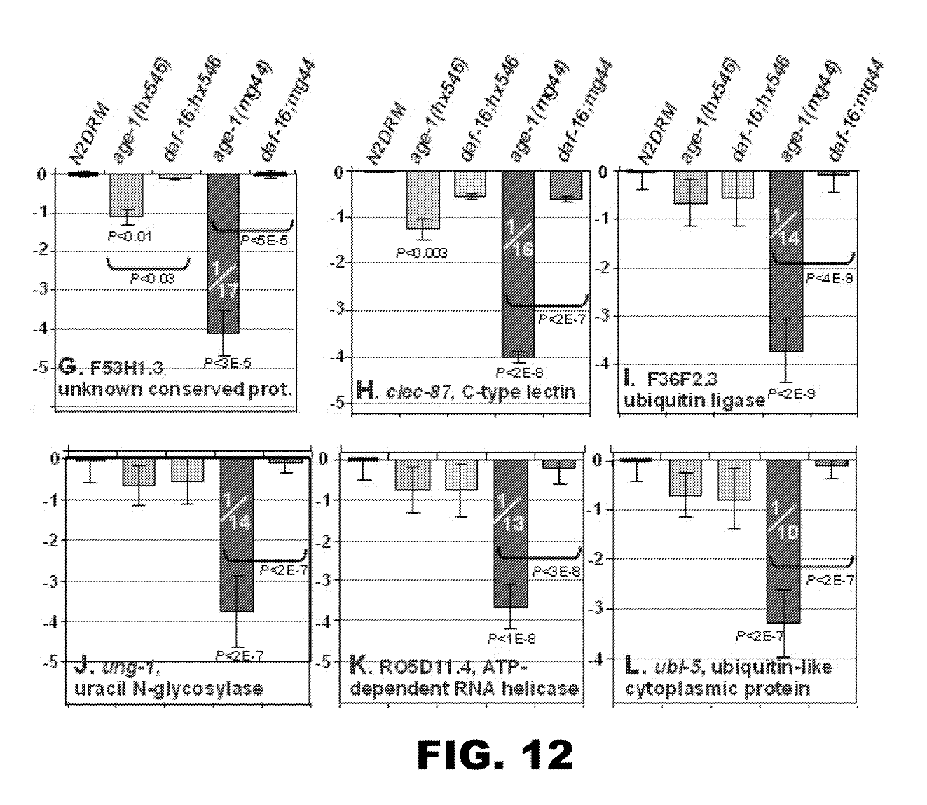

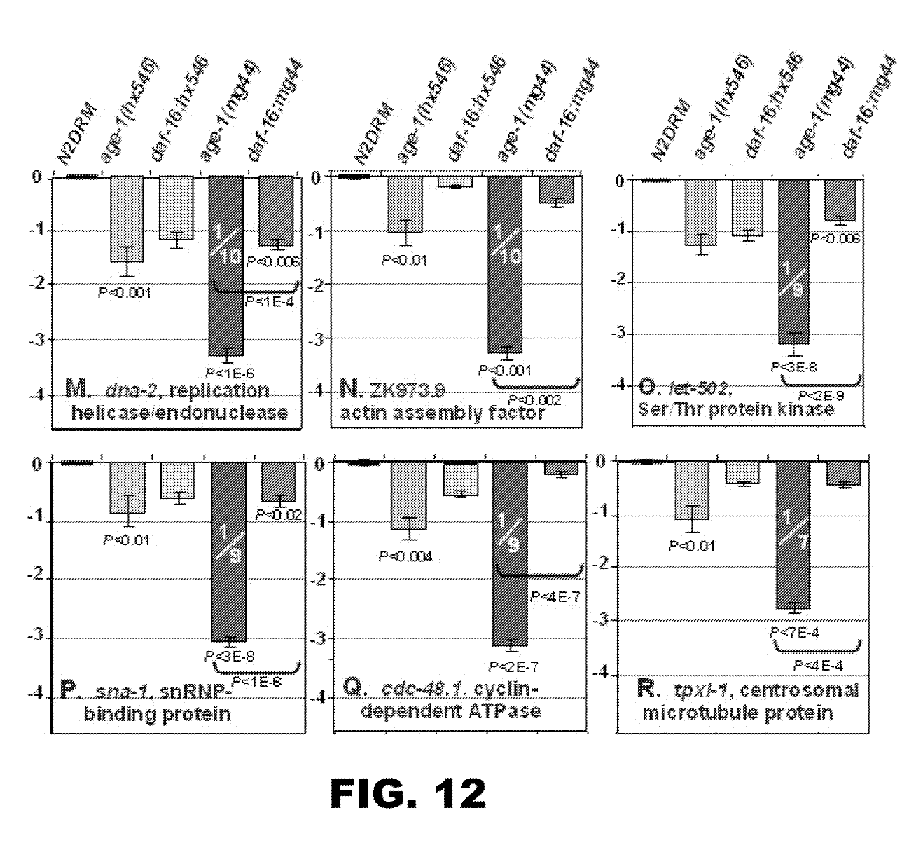

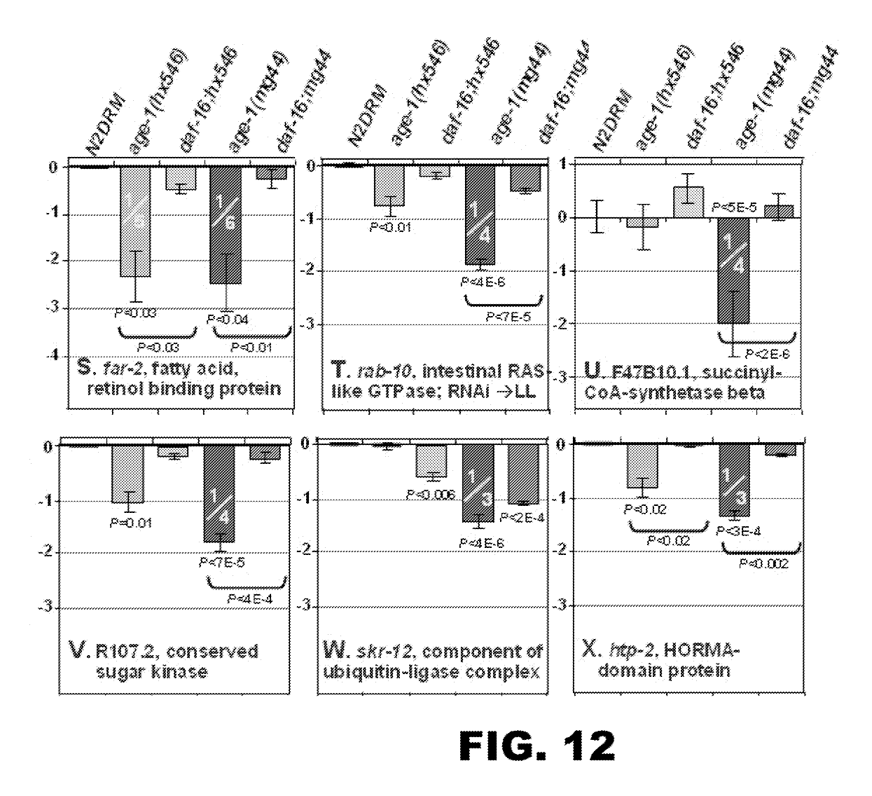

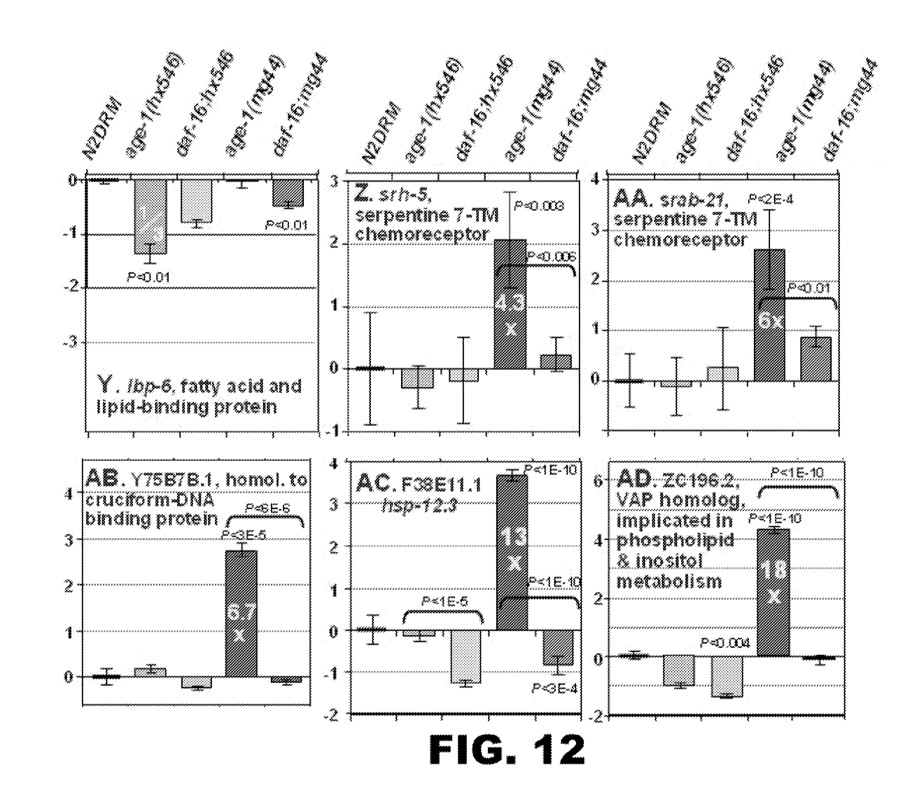

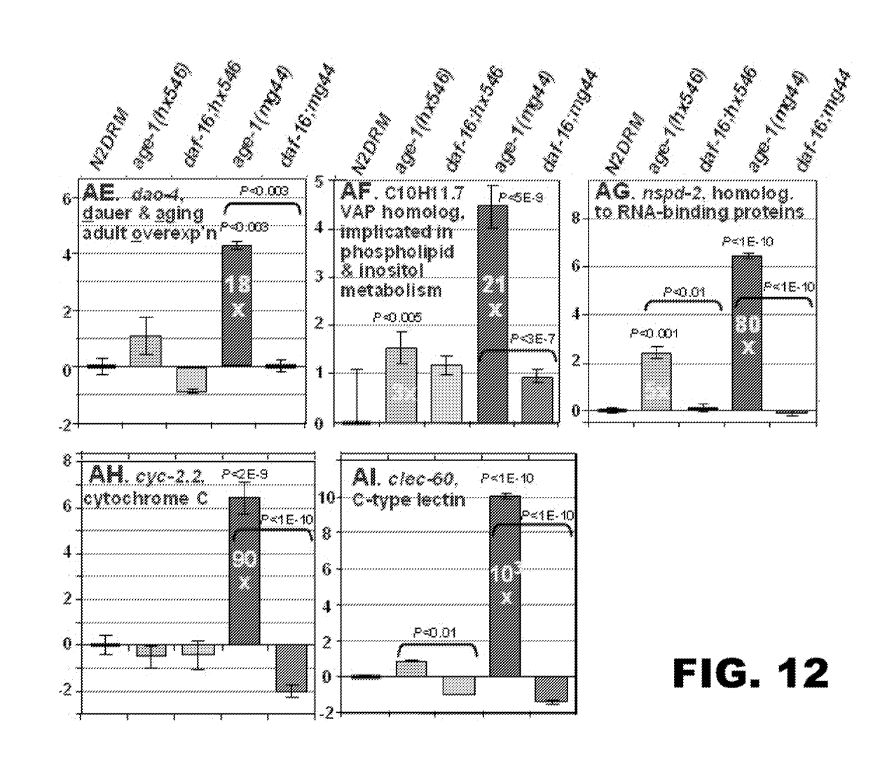

FIG. 12 presents transcript levels as determined by real-time PCR for 35 genes (as indicated in panels A-Z, AA-AI) initially selected based on differential cDNA hybridization to full-genome C. elegans microarrays. Means±SEM of steady-state transcript levels are shown on a log(2) scale—reflecting their measurement as threshold cycle numbers. Transcript levels were determined in wild-type N2DRM worms and age-1 mutant strains (alleles hx546 and mg44, each alone or in combination with a daf-16 mutation) at day-6 of adulthood. RT-PCR was performed for two cDNA syntheses for each of 3 or 4 independent biological replicates, normalized to the mean of two internal control genes that did not alter significantly among the strains. Significance: P values are shown for 2-tailed t-tests, regardless of whether the direction of change was predicted, comparing age-1(mg44) second-generation homozygotes to N2DRM (numbers just above age-1 error bars, without brackets), or to daf-16; age-1(mg44) double mutants (numbers over brackets). All t-tests used an N of 3 or 4, the number of biological replicates.

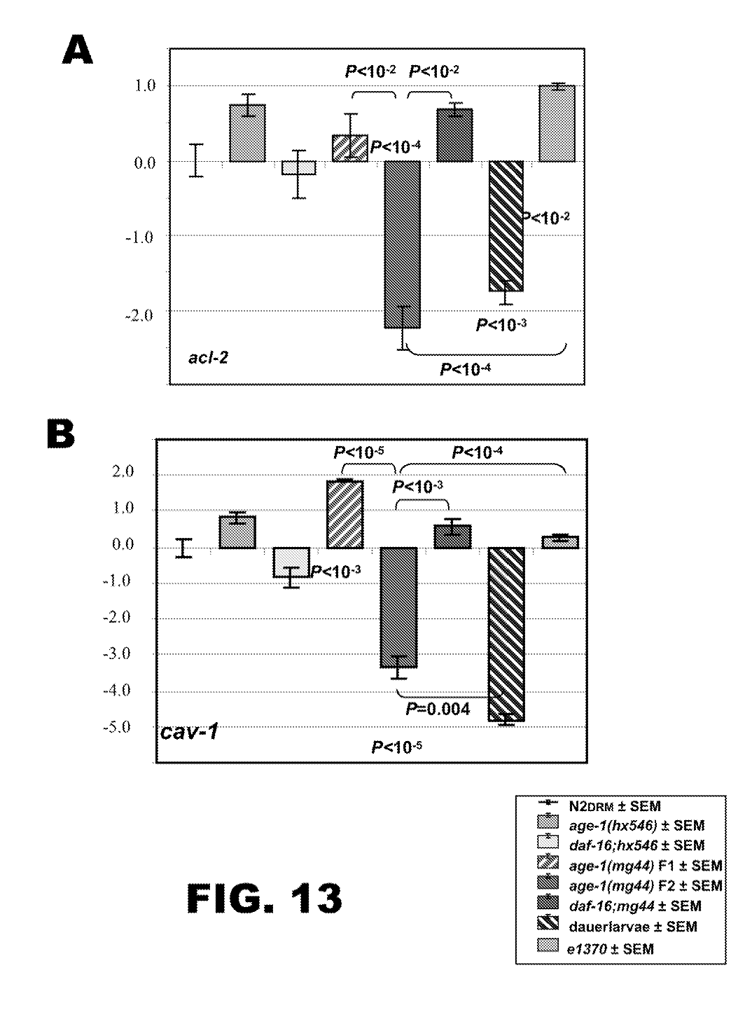

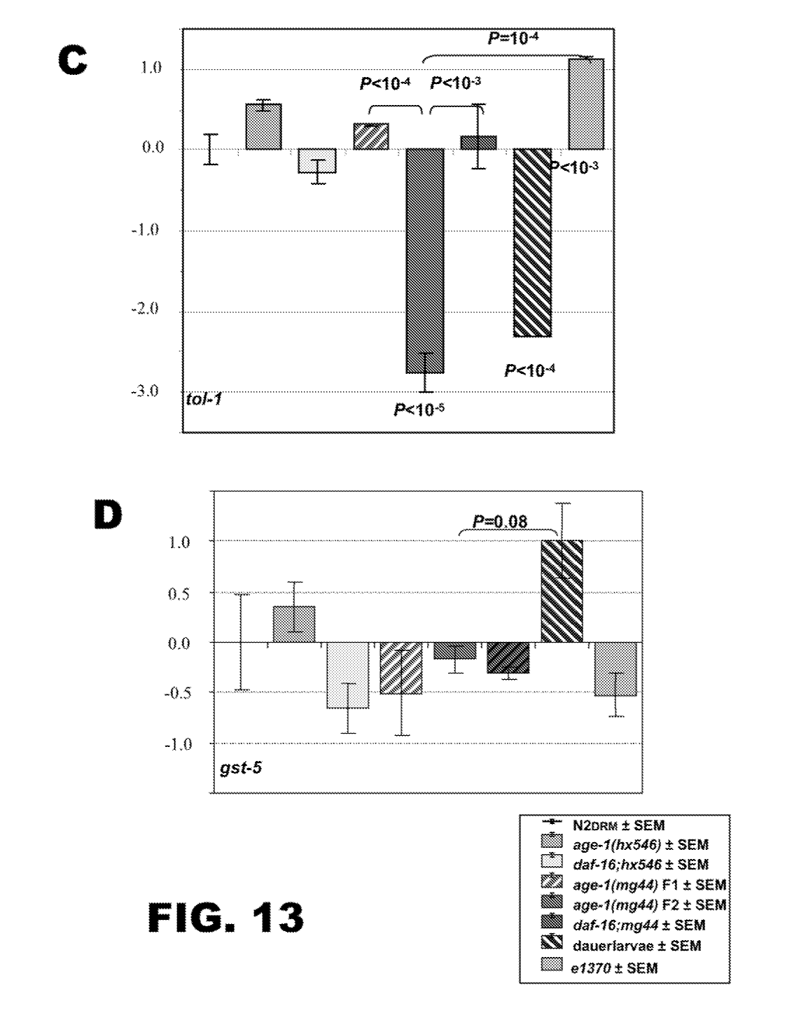

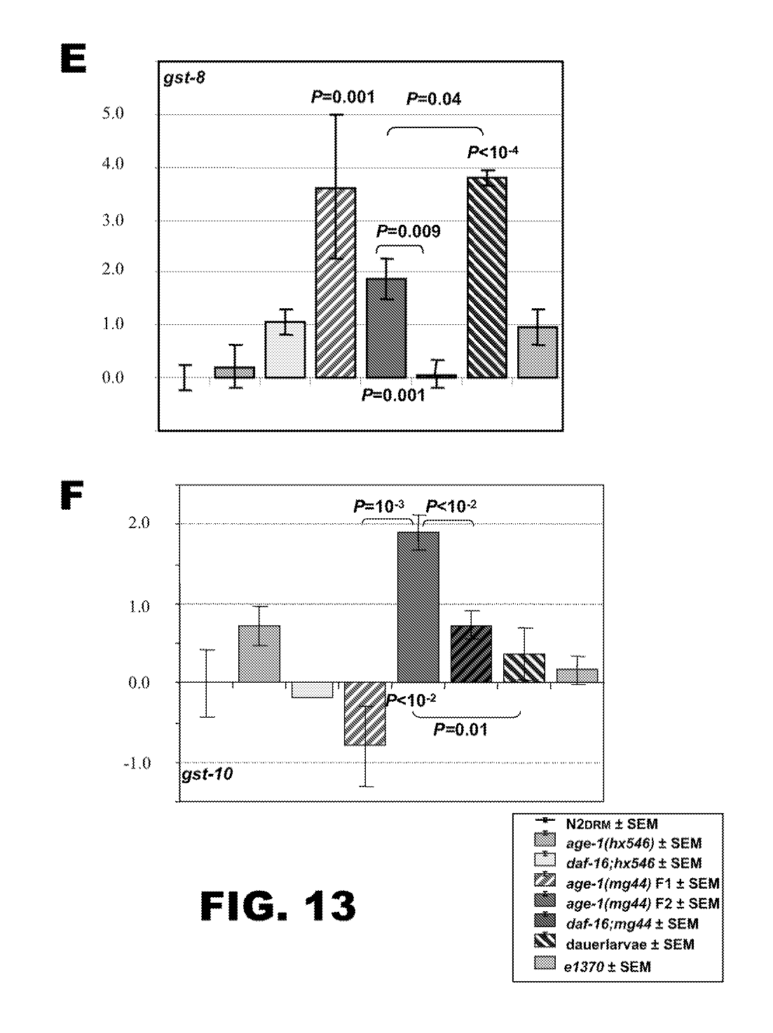

FIG. 13 presents transcript levels as determined by real-time PCR for (A) acl-2, a homolog of human AGPAT-alpha, (B) cav-1, a unique worm caveolin gene, (C) tol-1, the worm TLR, (D) gst-5, (E) gst-8, and (F) gst-10 in the different strains. Means±SEM are shown on a log₂ scale for steady-state transcript level in wild-type N2DRM worms, age-1 mutant strains, and dauer larvae. Statistics and significance are as detailed above.

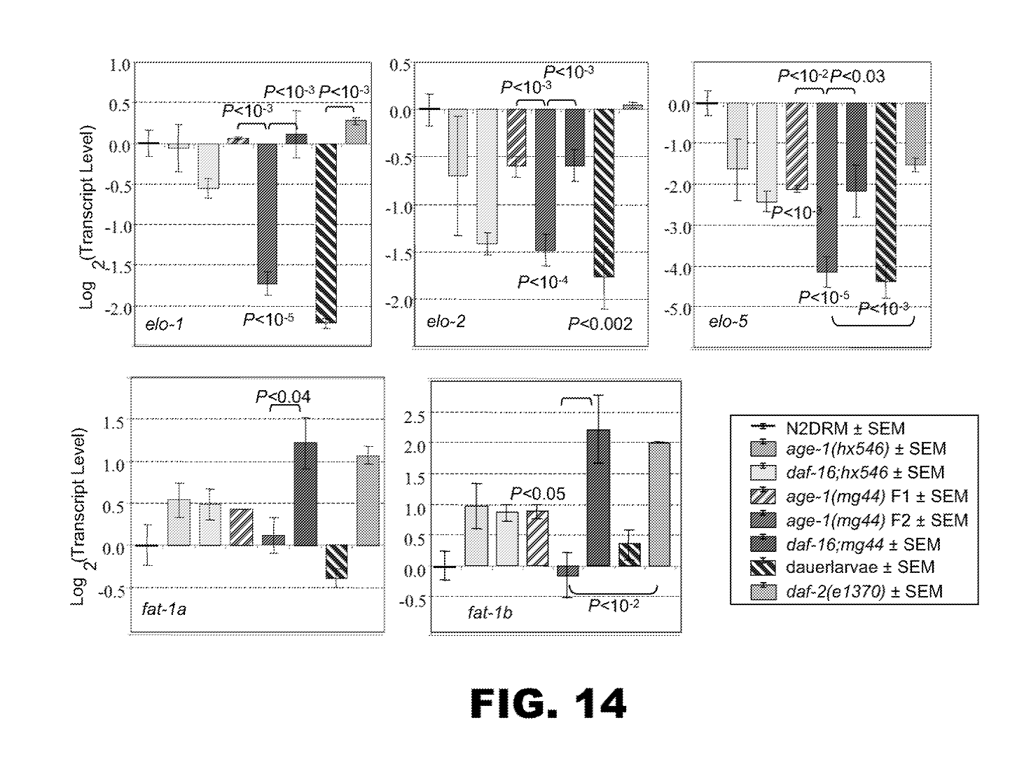

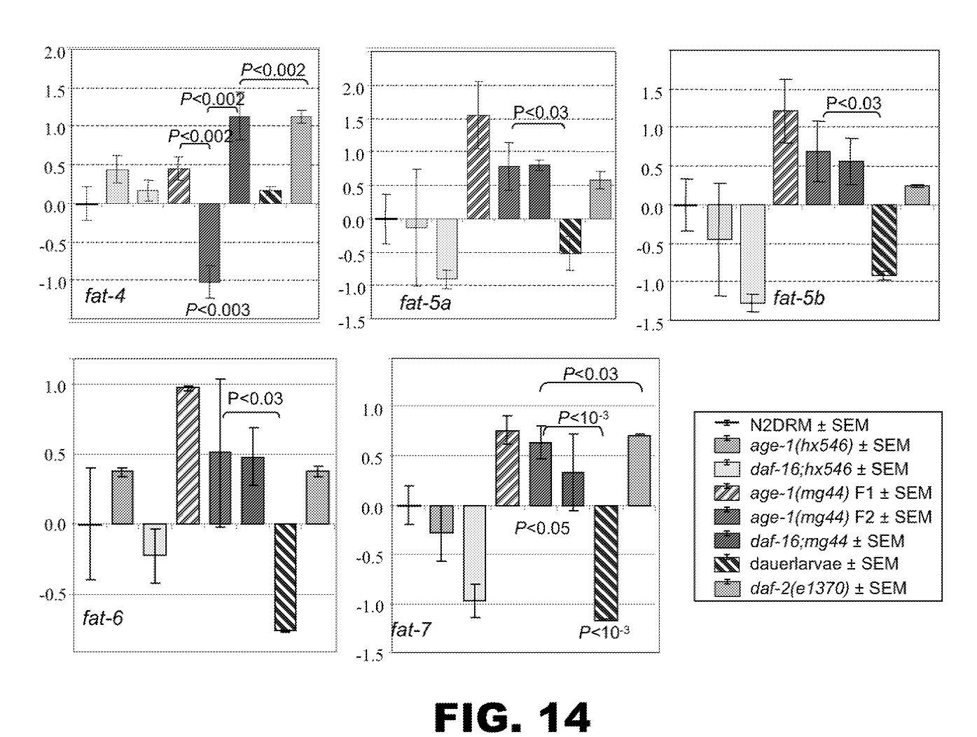

FIG. 14 presents transcript levels as determined by real-time PCR for ten lipid biosynthesis genes. Means±SEM are shown on a log₂ scale for steady-state transcript level in the different strains. Statistics and significance are as detailed above.

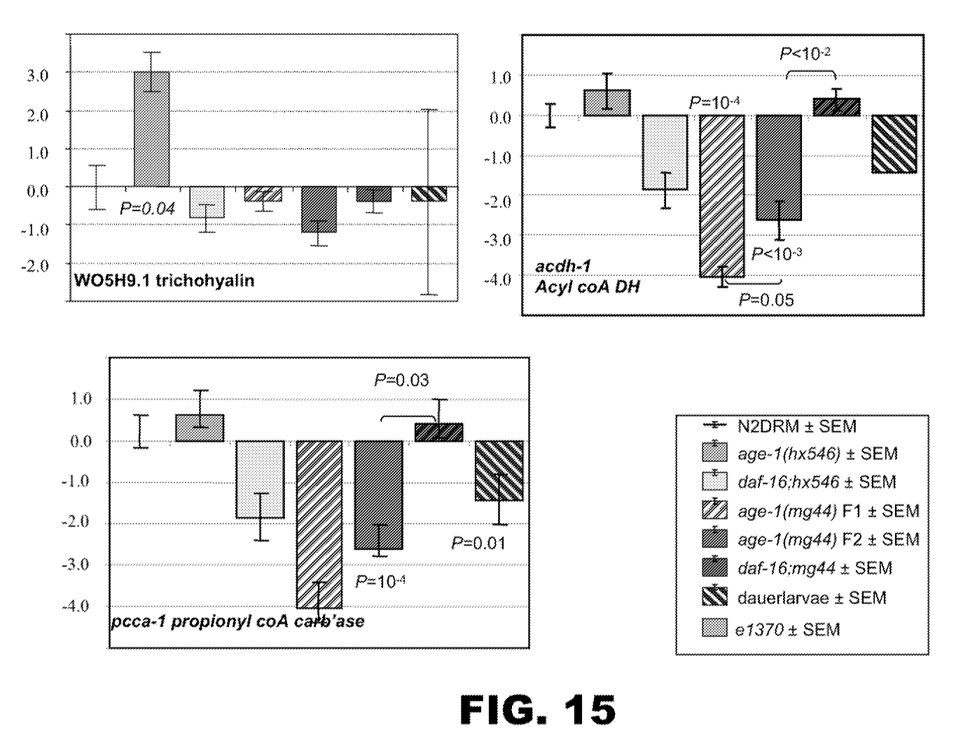

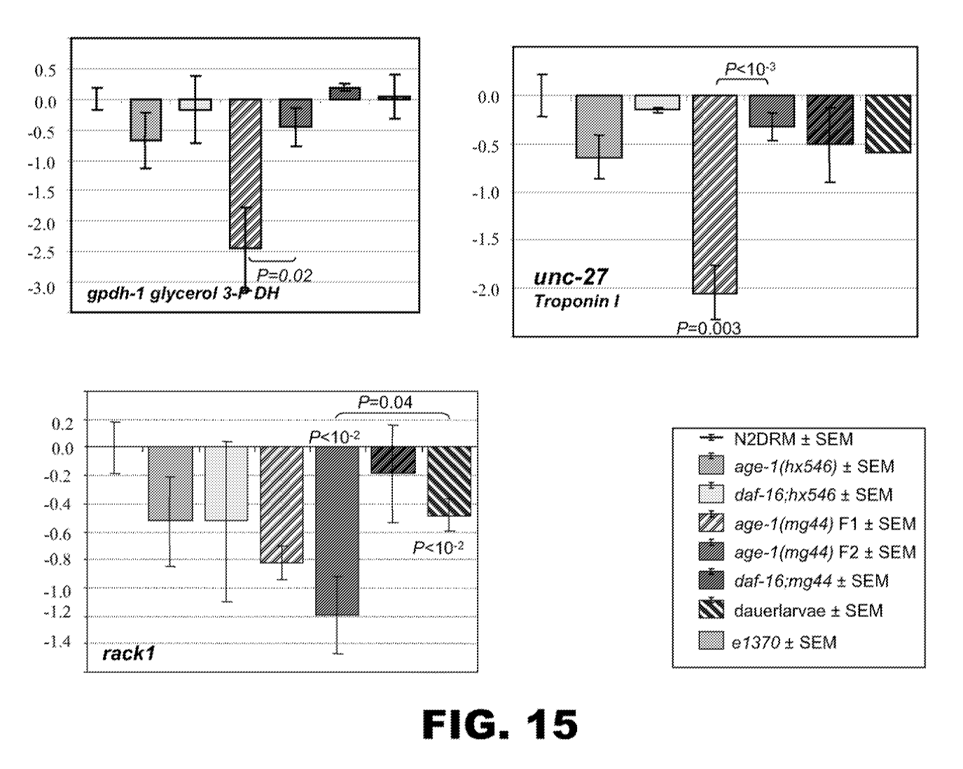

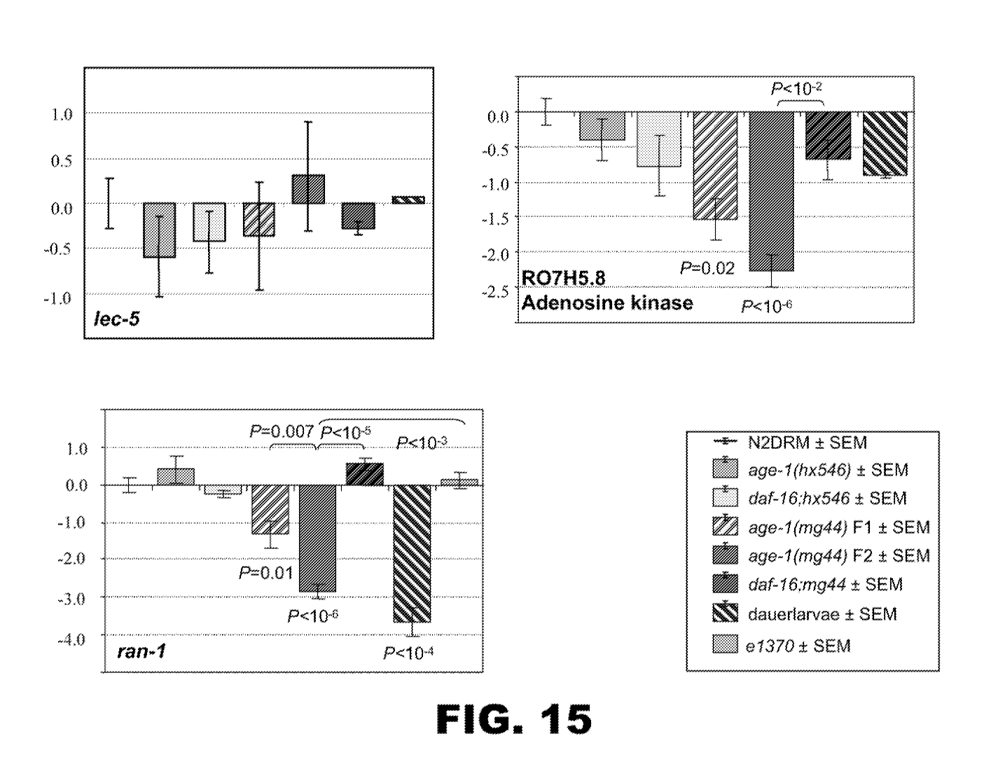

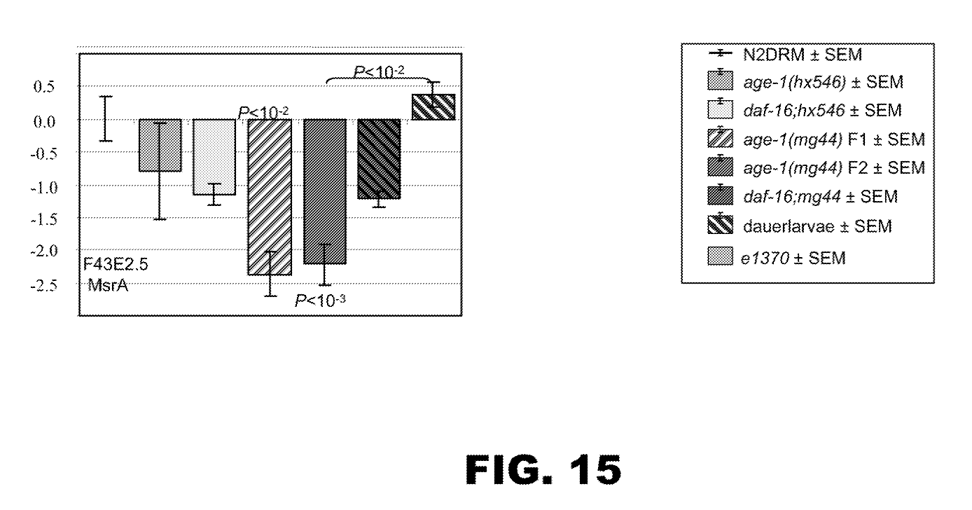

FIG. 15 presents transcript levels as determined by real-time PCR for ten other genes whose protein levels differ in the long-lived age-1 mutants relative to other strains. Means±SEM are shown on a log₂ scale for steady-state transcript level in the different strains. Statistics and significance are as detailed above.

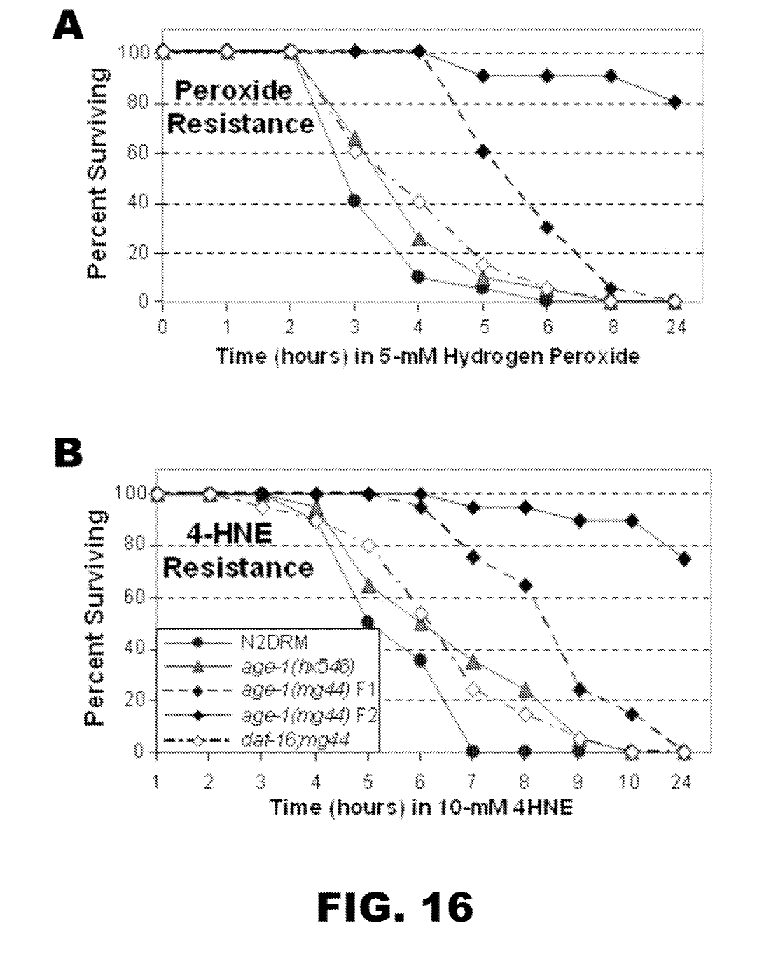

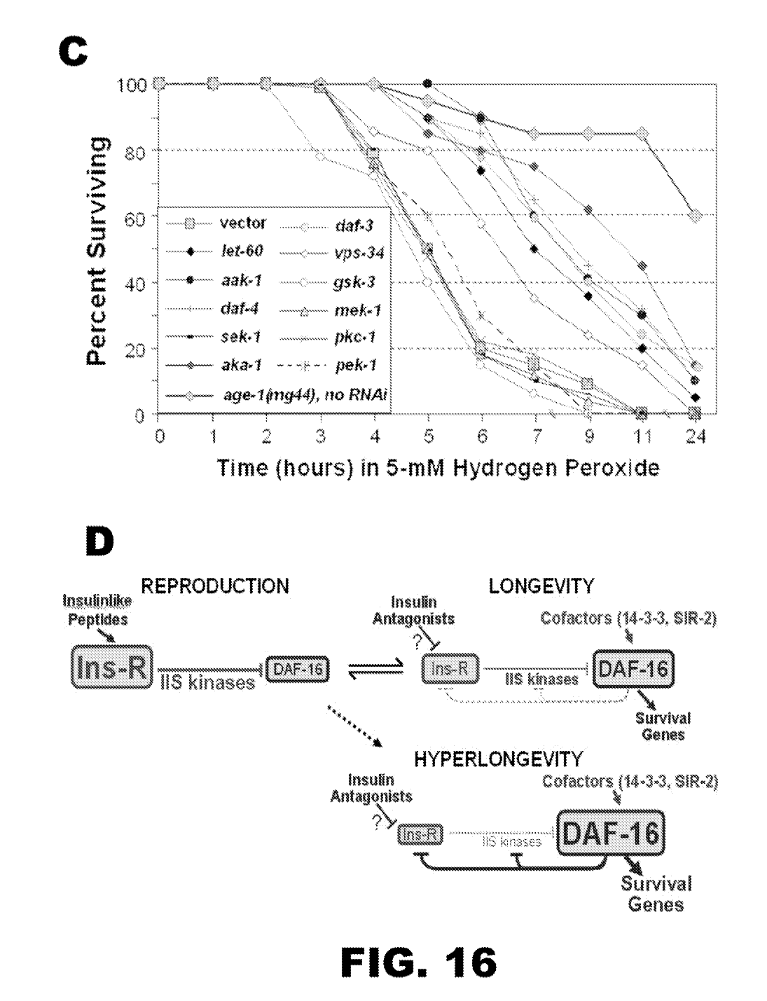

FIG. 16 depicts the survival of oxidative and electrophilic stresses. Survivals in (A) 5 mM hydrogen peroxide (H₂O₂), or (B) 10 mM 4-hydroxynonenal (4-HNE), were initiated at adult day 2 and maintained in liquid medium, 20±0.3° C., without bacteria. Results were replicated in independent experiments. Statistics (log-rank test, each n=40-50, both A and B): age-1(mg44) at F2 vs. F1 generation: p<10⁻⁷; age-1(mg44) F2 vs. any other strain: P<10⁻⁹; age-1(mg44) F1 vs. any other strain: p<10⁻³; age-1(hx546) vs. N2DRM: P<0.004; daf-16(mu86); age-1(mg44) vs. N2DRM: P<0.005. (C) Functional consequences of suppressing genes down-regulated in age-1(mg44). N2DRM worms were maintained on RNAi-expressing E. coli, at 20° C., on days 3-6 of adulthood. RNAi extended 60^{th}-percentile H₂O₂ survival, over N2DRM (significance, Gehans log-rank test), as follows: vps-34, 26% (P<0.03); let-60/RAS, 40% (P<0.001); daf-3, 49% (P<0.0003); aak-1, 49% (P<0.00003); daf-4, 57% (P<0.0001); aka-1, 96% (P<0.0002). All significant effects except vps-34 were confirmed (each P<0.001) in independent experiments. Peroxide survival of F2 age-1(mg44) day-62 adults, without RNAi, exceeded N2DRM 5.6-fold (P=10-6). (D) Model of the IIS “molecular switch”. Two upper diagrams depict alternative, self-reinforcing states that favor reproduction or longevity. State transitions are normally triggered by signaling modulators (e.g., insulin-like peptides or SIR-2/14:3:3 complex), to which age-1(mg44) F2 mutants (“hyperlongevity” state) cannot respond.

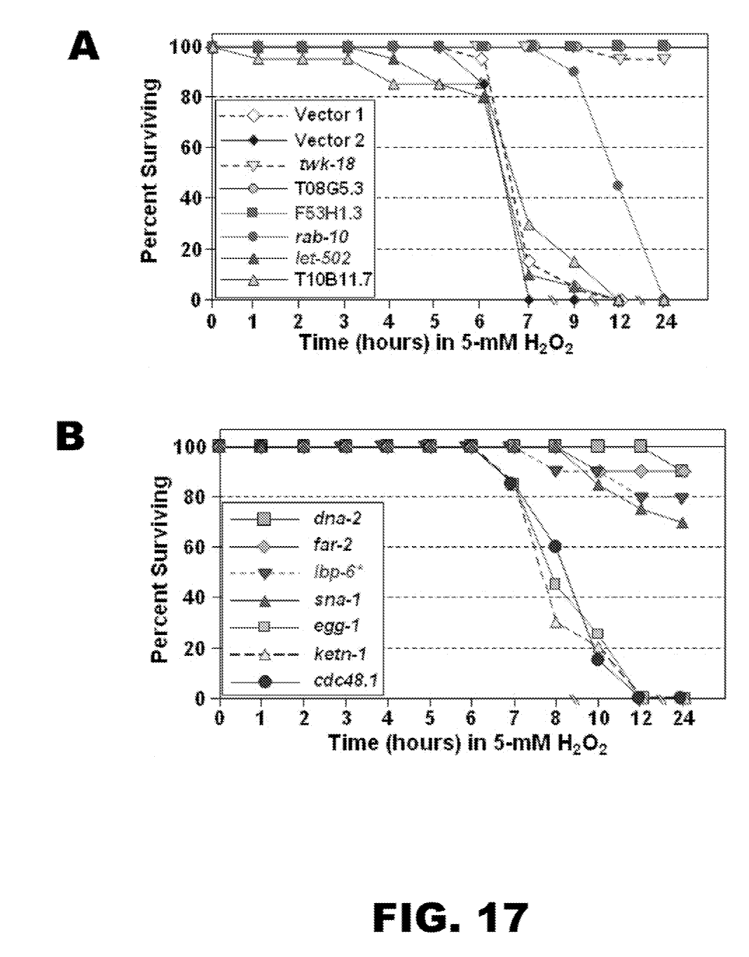

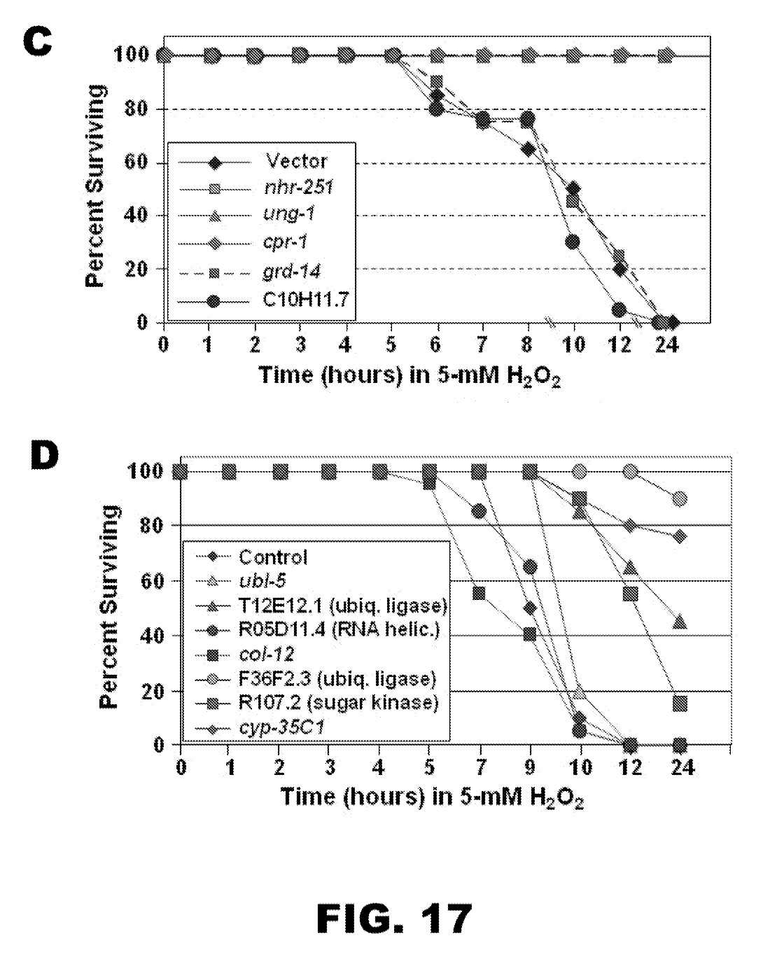

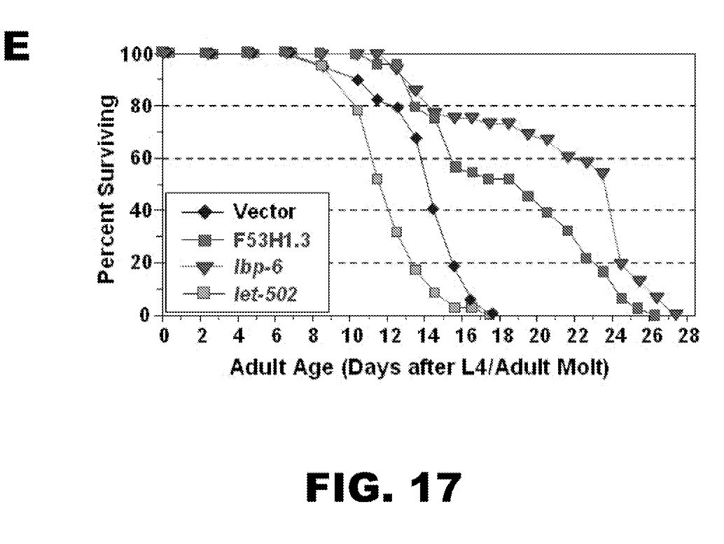

FIG. 17 illustrates that RNAi treatments confer resistance to hydrogen peroxide toxicity, and can extend life. N2DRM worms were fed bacteria expressing the indicated RNAi, from the L4/adult molt, either (A-D) for 2-3 days prior to testing resistance to 5 mM H₂O₂, or (E) throughout the survivals. RNAi treatments significantly extending survival in hydrogen peroxide (and P values determined by log-rank test) were (A) twk-18, T08G5.3, F53H1.3 (each P<10⁻⁶), and rab-10 (P<0.001); (B) dna-2, far-2, Ibp-6, and sna-1 (each P<10⁻⁵); (C) nhr-251, ung-1, and cpr-1 (each P<10⁻⁶); (D) T12E12.1, R107.2 (each P<0.001), cyp-35C1 (P<10⁻⁴), F36F2.3 (P<10⁵); (E) F53H1.3, Ibp-6 (each P<10⁻⁶). All significant increases in survival indicated were confirmed in

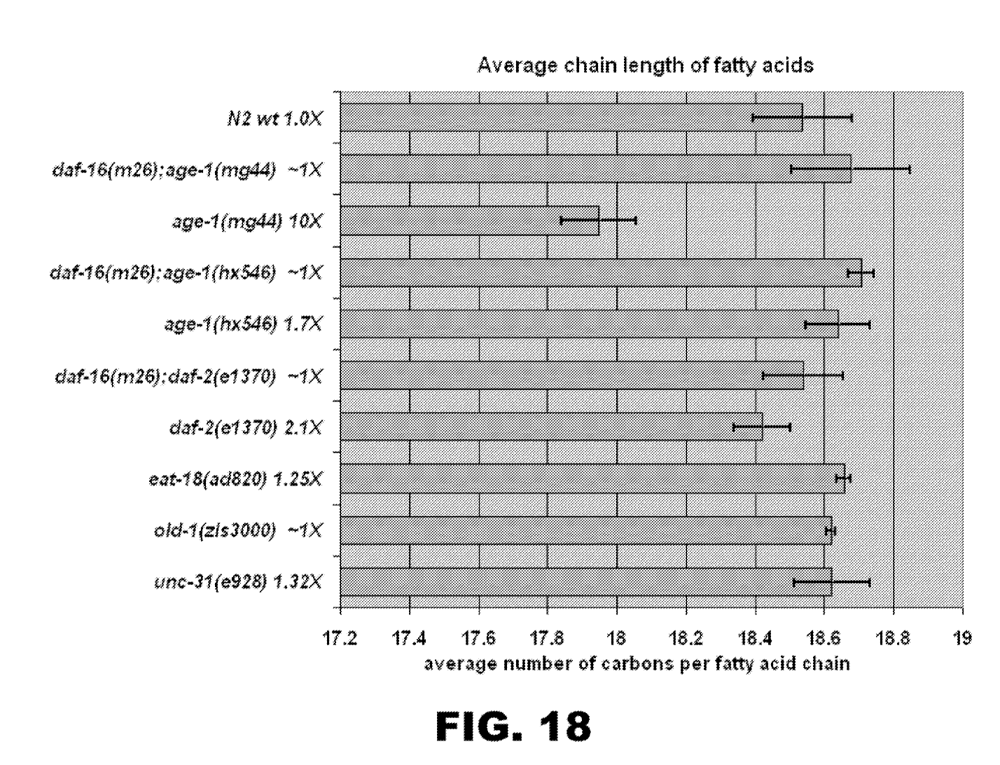

FIG. 18 shows that age-1(mg44) mutants have lipids with shorter chain lengths, on average. Plotted is the average number of carbons per fatty acid chain for lipids extracted from each of the indicated strains. * P=0.0009 vs. N2(wt) by 2-tailed t-test. Error bars show±1 standard deviation.

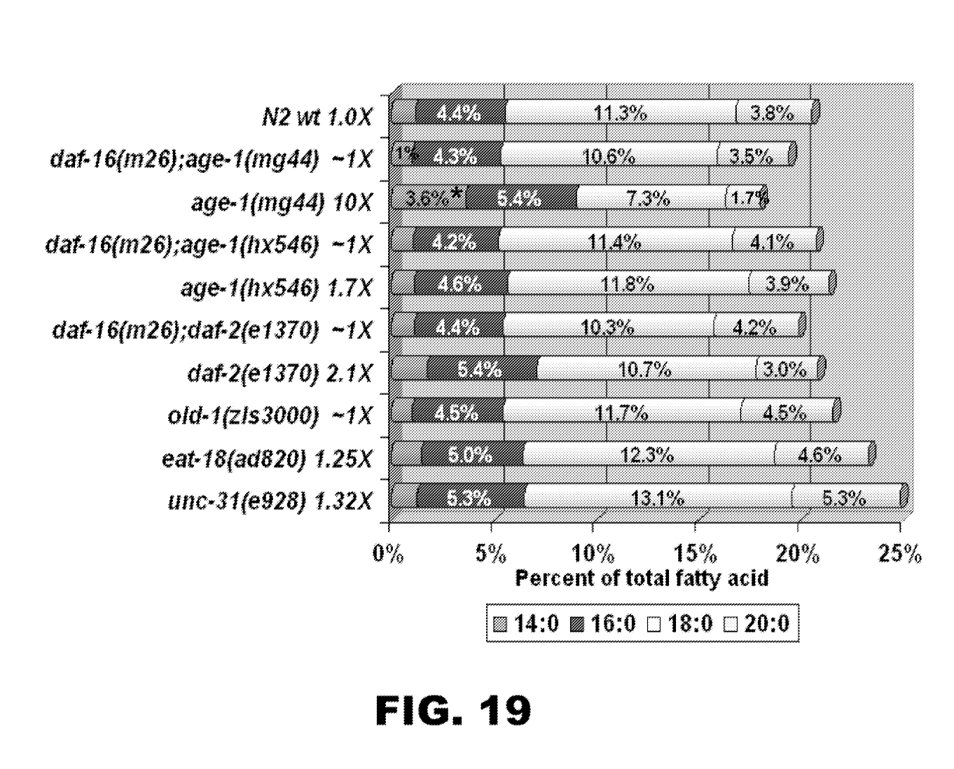

FIG. 19 illustrates the altered profile of saturated fatty acids in age-1(mg44) mutants. Plotted is the percent of total fatty acid for myristic acid (14:0), palmitic acid (16:0), stearic acid (18:0), and eicosanoic acid (20:0) for each of the indicated strains. RLS=relative lifespan. * P=0.034 for 14:0. † P=0.044 for 20:0 each vs. daf-16;age-1(mg44), by 2-tailed t-tests, assuming heteroscedasticity.

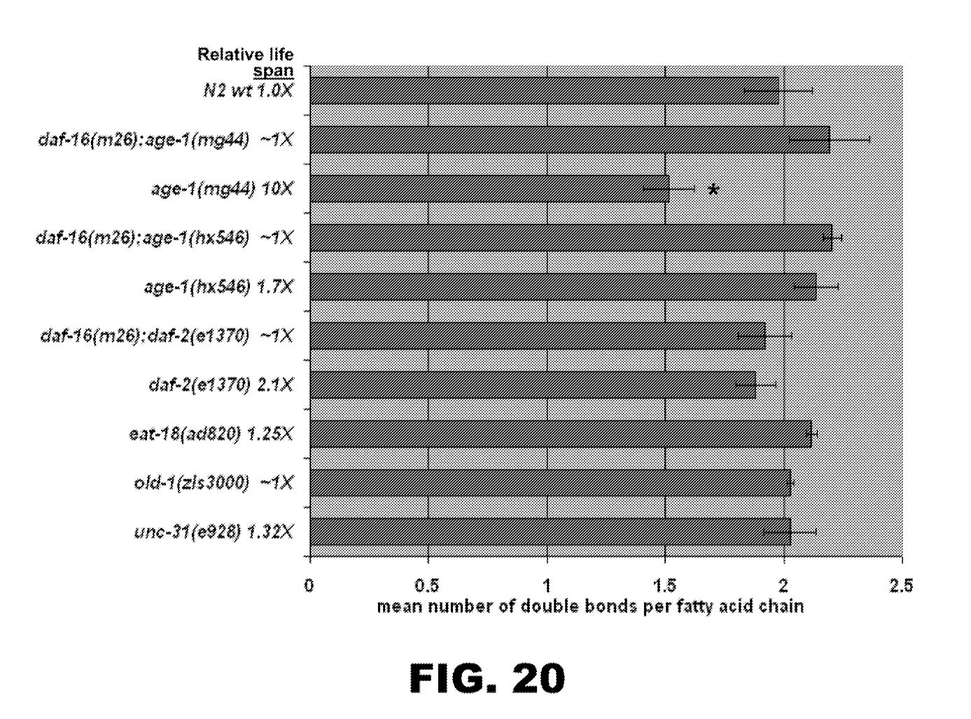

FIG. 20 illustrates that the lipids of age-1(mg44) mutants have fewer double bonds than the lipids of other strains. Plotted is the mean number of double bonds per fatty acid chain for each of the indicated strains. *P=0.0009 vs. N2(wt), by 2-tailed t-test. Error bars show±1 standard deviation.

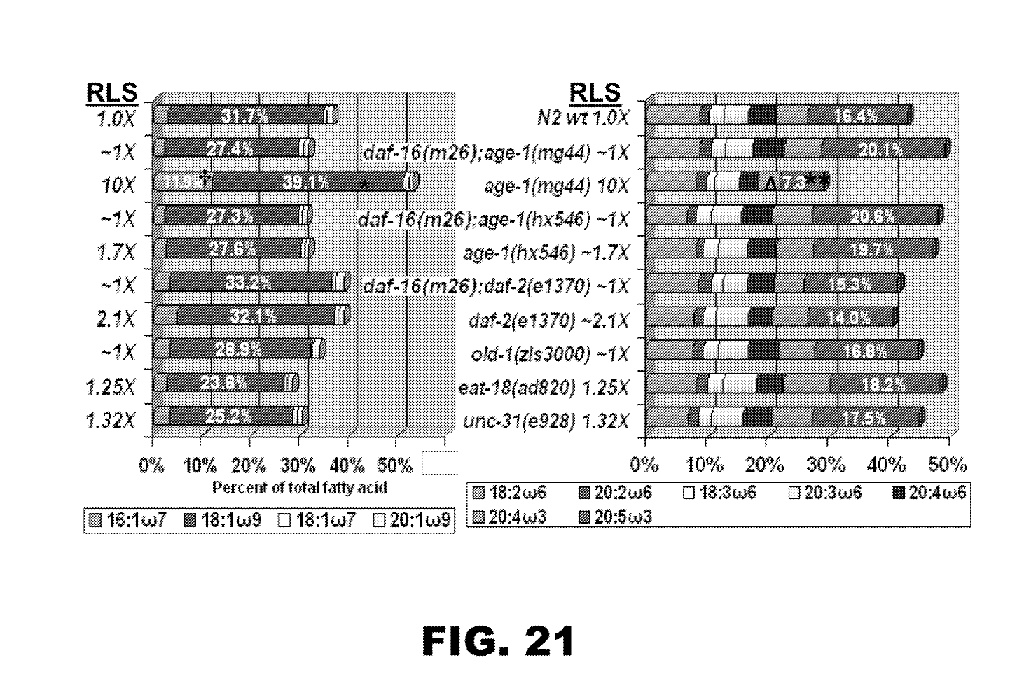

FIG. 21 shows the relative amounts of monounsaturated (left) and polyunsaturated (right) fatty acids in lipids extracted from different worm strains. ** P=5×10⁻⁵; Δ P=0.008; *P=0.0014; and † P=0.0012 by a 2-tailed t-test intended for heteroscedastic distributions, in comparison with the daf-16;age-1(mg44) strain.

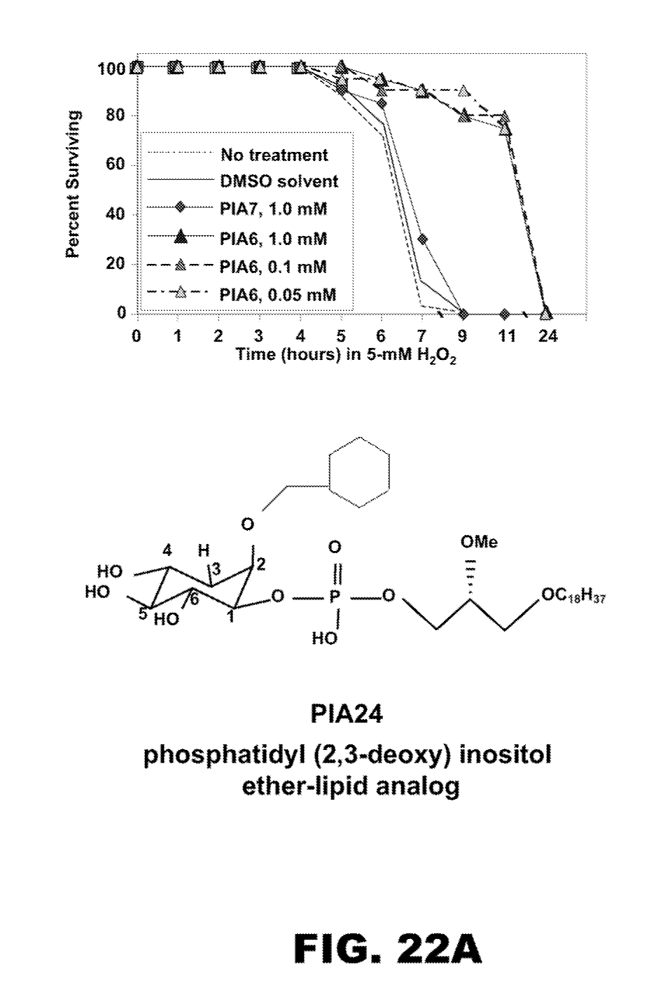

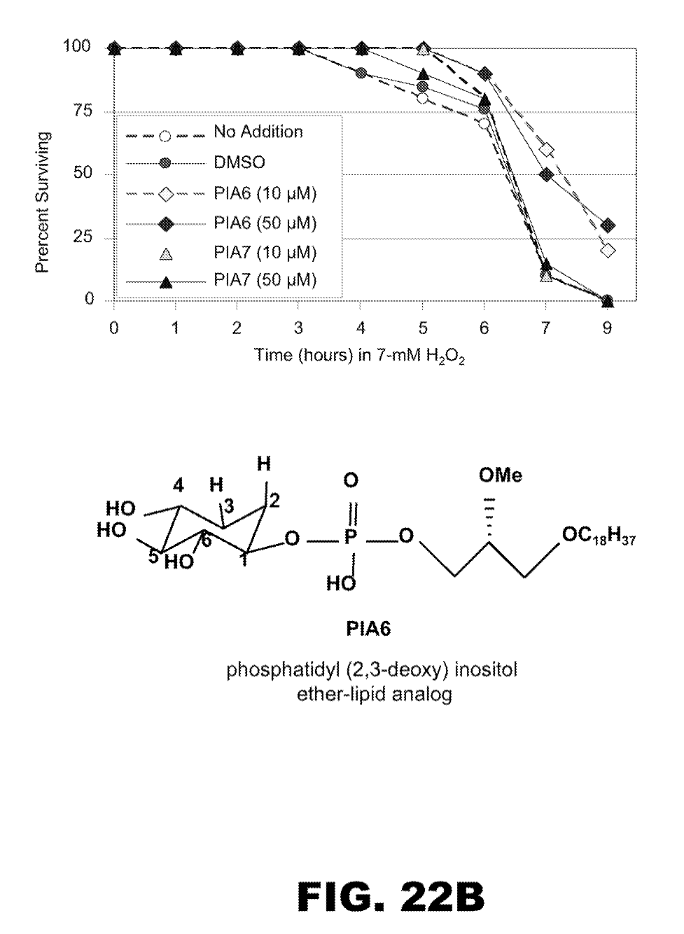

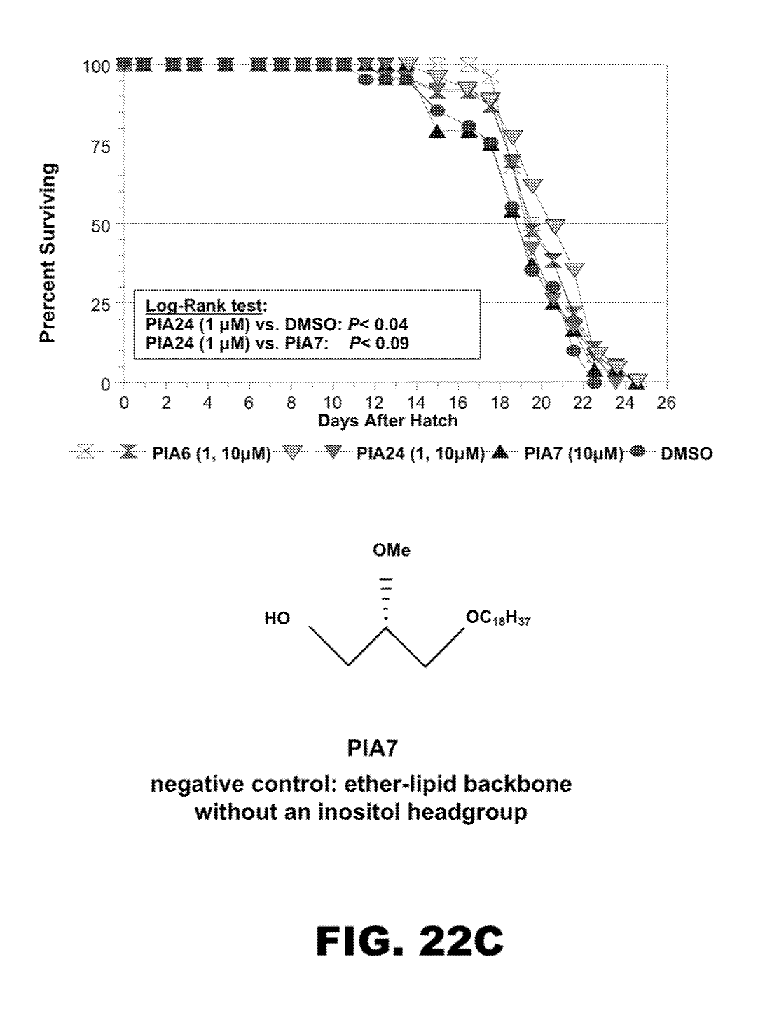

FIG. 22 illustrates that phosphatidylinositol analogs, unable to be phosphorylated at position 3, confer resistance to hydrogen peroxide toxicity, and extend lifespan. (A, B) PIA6 increased survival in 5 mM or 7 mM H₂O₂, respectively. (C) PIA24 increased survival relative to the inactive analog, PIA7, or DMSO.

### DETAILED DESCRIPTION OF THE INVENTION

The present invention provides methods for identifying agents that modulate lifespan and resistance to oxidative and/or electrophilic stress, and methods for identifying biomarkers of longevity or PIP3 response pathways that govern longevity. It has been discovered that the phenotype of a second (and later) generation nematode homozygous for age-1(mg44) null or age-1(m333) null mutations differs markedly from a first generation age-1(mg44), a first generation age-1(m333), or an age-1 mutant with a weaker allele (e.g., hx546) at any generation. The lifespan of a second generation homozygous age-1 null mutant nematode is about ten times longer than that of a wild type nematode and about four times to about five times longer that that of a first generation age-1(mg44) or age-1(m333) mutant, or an age-1(hx546) mutant. Furthermore, it has been discovered that the extremely long-lived second-generation age-1 null nematode has increased resistance to oxidative and/or electrophilic stresses relative to wild type or other age-1 mutants. Thus, resistance to oxidative and/or electrophilic stress is an excellent surrogate measure of lifespan.

The invention provides a method of screening for agents that provide protection against oxidative and/or electrophilic stresses. Changes in the stress response of a test nematode will be compared to those of a negative control nematode and the extremely long-lived age-1 null nematode. Thus, a change in the stress response of the test nematode away from that of a negative control nematode and towards that of a long-lived mutant nematode indicates that the agent increases resistance to oxidative and/or electrophilic stress. An agent that provides increased protection against oxidative and/or electrophilic stress may also extend lifespan.

Also provided are methods for identifying biomarkers of longevity or pathways governing longevity in response to PIP3 signaling. These methods take advantage of the phenotypic differences between the long-lived age-1 null nematodes and wild type or other age-1 mutants to tease apart the molecules and the pathways that respond to PIP3 signaling to confer the greatly extended lifespan and increased stress resistance to the long-lived age-1 null nematodes. Once longevity biomarkers or pathways governing longevity in response to PIP3 signaling are identified, these will lead to the development of therapeutic interventions to extend lifespan or slow the aging process.

I. Method For Identifying Agents that Modulate Resistance to Oxidative and/or Electrophilic Stress

Provided herein is a method for identifying an agent that modulates resistance to oxidative and/or electrophilic stress. As detailed above, genetic changes that most profoundly extend lifespan also markedly increase resistance to both oxidative and electrophilic stresses; thus, resistance to an oxidative and/or electrophilic stress may be used as a surrogate measure of lifespan. The method comprises contacting a test nematode with at least one test agent that may provide protection against an oxidative and/or electrophilic stress. The test nematode, a negative control nematode, and an extremely long-lived age-1 null mutant nematode are then exposed to at least one oxidative and/or electrophilic stress. After a suitable period of time, responses to the oxidative and/or electrophilic stress are measured in the test nematode, the negative control nematode, and the mutant nematode. The responses may be measured at the physiological or molecular levels. The responses are then compared to determine whether the agent modulated resistance to stress. An agent that does not change the stress response of the test nematode relative to the negative control nematode is not a modulator of resistance to oxidative and/or electrophilic stress. A change in the stress response of the test nematode away from that of the negative control nematode and towards that of the extremely long-lived mutant nematode indicates that the agent increases resistance to oxidative and/or electrophilic stress. A change in the stress response of the test nematode away from both that of the negative control nematode and that of the extremely long-lived mutant nematode indicates that the agent decreases resistance to oxidative and/or electrophilic stress.

a. Nematodes

Nematodes are elongated symmetrical roundworms that constitute one of the largest and most successful phyla in the animal kingdom. Some nematode species are free-living, i.e., they live in soil or water and feed on bacteria, algae, fungi or other matter. Other nematode species are parasitic, infecting plants, invertebrates, and vertebrates, including humans.

A variety of nematodes species may be used in the present invention. The nematode may be a parasitic species, such as those in the Strongylida, Rhabditida, Ascaridida, Spirurida, Oxyurida, Enoplida, Tylenchida, or Dorylaimida orders. Alternatively, the nematode may be a free-living species. In one embodiment, the nematode may be a free-living species of the genus Caenorhabditis. In a preferred embodiment, the nematode may be of the species Caenorhabditis elegans. Suitable stains of nematodes may be acquired from the Caenorhabditis Genetics Center (CGC; Minneapolis, Minn.). Methods to culture and propagate C. elegans are well known in the art.

i. Test Nematode and Negative Control Nematode

The test nematode and negative control nematode are substantially identical, i.e., have the same genotype and are of the same approximate age. The test nematode and negative control nematode may be wild type organisms. Alternatively, the test nematode and the negative control nematode may be mutants, whereby the genetic material is altered such that the production of a gene product is perturbed. The mutation may be naturally occurring (e.g., due to copying errors during replication), or it may be induced by exposure to ultraviolet or ionizing radiation, chemical mutagens, or viruses. Suitable mutants include age-1(hx546) or daf-2(e1370) homozygous mutants. Lastly, the test nematode and the negative control nematode may also be transgenic organisms. A transgenic organism refers to one in which at least one cell of the organism contains a heterologous nucleic acid introduced by any of a variety of techniques well known in the art. The heterologous nucleic acid may be extrachromosomal or it may be integrated into a chromosome of the organism. An example of a suitable transgenic animal is one carrying a reporter gene, such as green fluorescent protein (GFP), which may be employed as a visible reporter of oxidative or electrophilic stress or responses thereto, in particular when fused to another gene or domain, such as the sequence encoding a plextrin-homology domain.

ii. Mutant Nematode

The mutant nematode is an extremely long-lived age-1 null mutant. Mutations in the age-1 gene were first described in a genetic screen for increased longevity (Klass, Mech. Ageing Dev. (1983) 22:279-286; Friedman and Johnson, Genetics (1988) 118:75-86), and were further characterized (Gottlieb and Ruvkun, Genetics (1994) 137:107-120; Tissenbaum and Ruvkun, Genetics (1998) Genetics 148:703-717; U.S. Pat. No. 7,094,889), all of which are hereby incorporated by reference in their entirety. The above-cited studies reported that age-1 homozygous null mutants arrested as dauer larva (when grown at about 25° C.), and failed to mature to adults within the times allowed (typically 50 hr) at lower temperatures.

It has been discovered (see the examples) that age-1(mg44) and age-1(m333) homozygous null mutant nematodes develop into adults when grown for sufficient time at temperatures lower than about 25° C. (e.g., about 15° C. to about 20° C.), and that second generation homozygous null mutant adults have greatly extended lifespans. The age-1(mg44) and age-1(m333) mutant nematodes, as second generation homozygotes, have adult lifespans that range from about seven times to about twelve times longer than those of wild type nematodes and range from about four times to about five times longer than those of the identical mutants in the first generation, or other age-1 mutants at any generation. It should be noted, that once age-1(mg44) and age-1(m333) mutants have developed into adults, they may be grown at higher temperatures, such as about 25° C.. Additionally, it has been discovered (see examples) that the age-1(mg44) and age-1(m333) adults exhibit about three-fold to about eight-fold longer survival in the presence of oxidative and/or electrophilic stresses than wild type animals. Thus, the extremely long-lived age-1 mutants are excellent positive controls in screens that seek to identify agents that modulate lifespan and resistance to oxidative and/or electrophilic stress. Other long-lived mutants that may be used as positive controls include age-1 null mutants and age-1 truncation mutants that may be created or discovered.

The test nematode, the negative control nematode, and the mutant nematode used in a screening method will generally be of the same approximate age. The age of the nematodes can and will vary depending upon the nematodes used and the agent being tested. In one embodiment, the test, negative control, and mutant nematodes may be day-1 adults. In another embodiment, the test, negative control, and mutant nematodes may be day-2 adults. In an alternate embodiment, the test, negative control, and mutant nematodes may be day-3 adults. In an alternate embodiment, the test and negative control nematodes may be postgravid (e.g., day-8) adults, to more closely resemble the infertile second-generation age-1 null (mg44 or m333) homozygous mutant nematodes. The post gravid adults may be wild type nematodes, or they may be moderately long-lived mutant nematodes, such as age-1[hx546] or similar age-1 homozygous mutants, or daf-2(e1370) or similar daf-2 homozygous mutants. Alternatively, test and negative control nematodes may contain mutations preventing fertility, or in particular, oogenesis. In any of the above embodiments, different age-1 null mutations (other than mg44 or m333) may be substituted for those existing mutations, and such mutant nematodes, in the second generation, may be older in days (but not in percent of adult lifespan) than the control nematodes.

b. Oxidative and/or Electrophilic Stress

The method comprises exposing the test nematode, the negative control nematode, and the mutant nematode to an oxidative and/or electrophilic stress. Oxidative stress may be induced by any treatment that generates reactive oxygen species, such as hydroxy radicals, superoxide radicals, alkoxy radicals, peroxy radicals, hydrogen peroxide, organic hydroperoxide, hypochlorous acid, or peroxynitrite. Oxidative stress may be induced by contact with hydrogen peroxide. Alternatively, oxidative stress may be induced by contact with the herbicide, paraquat (also known as methyl viologen), which generates superoxide anions. One skilled in the art will appreciate that compounds related to paraquat, such as diquat, cyperquat, diethamquat, difenzoquat, and morfamquat, may also be used to induce oxidative stress. Electrophilic stress may be induced by contact with 4-hydroxynonenal, a lipoperoxidation end product, or other electrophilic agent such as acrolein, N-ethyl maleimide, or 1-chloro-2,4-dinitrobenzene (CDNB). A combination of tests employing oxidative and electrophilic stresses, e.g., hydrogen peroxide and 4-hydroxynonenal, may also be used to identify agents conferring resistance to both oxidative and electrophilic stresses.

The concentration of the agent that induces oxidative and/or electrophilic stress can and will vary depending upon the agent, response time, temperature and other conditions of assay, and the robustness of the nematodes. As an example, the concentration of hydrogen peroxide may range from about 0.5 mM to about 50 mM, preferably from about 1 mM to about 10 mM, and most preferably from about 3 mM to about 5 mM. The concentration of paraquat may range from about 50 mM to about 500 mM, preferably from about 100 mM to about 200 mM, and most preferably about 150 mM. The concentration of 4-hydroxynonenal may range from about 2 mM to about 50 mM, preferably from about 5 mM to about 20 mM, and most preferably about 10 mM. Alternatively, a stabilized electrophile precursor compound such as 4-hydroxynonenal triacetate, 4-HNE(Ac)₃ may be substituted for 4-HNE, at lower concentrations (e.g., 0.3-2 mM). The stress-inducing agent may be included in the solid medium on which nematodes are grown, or it may be included in a liquid medium provided to the animals, or via bacteria or other particulate vehicles.

c. Test Agent

The test nematode is also contacted with at least one test agent. The stress response of the test nematode is compared to the stress responses of the negative control and mutant nematodes to determine whether the test agent modulates resistance to oxidative and/or electrophilic stress.

Non-limiting examples of suitable test agents include natural compounds, synthetic compounds, small organic compounds, peptides, peptide nucleic acids, peptidomimetics, antibodies, antisense oligonucleotides, aptomer oligonucleotide, or double stranded RNA interference molecules. Furthermore, the test agent may be an individual compound or it may be a member of a combinatorial chemical library. A combinatorial chemical library is generally a collection of diverse chemical compounds generated by either chemical synthesis or biological synthesis by combining a number of chemical “building blocks.” For example, a linear combinatorial chemical library such as a polypeptide library is formed by combining a set of amino acids in every possible way for a given compound length (i.e., the number of amino acids in a polypeptide compound). Millions of chemical compounds may be synthesized through such combinatorial mixing of chemical building blocks.

Preparation of combinatorial chemical libraries is well known to those of skill in the art. Methods of making combinatorial libraries include biological libraries; spatially addressable parallel solid phase or solution phase libraries; synthetic library methods requiring deconvolution; the ‘one-bead one-compound’ library method; and synthetic library methods using affinity chromatography selection. The above-cited methods have been used to synthesize libraries of peptides (see, e.g., U.S. Pat. No. 5,010,175; Furka, 1991, Inmt. J. Pept. Prot. Res. 37:487-493; Houghton et al., 1991, Nature 354:84-88); peptoids (PCT Publication No. WO 91/19735); encoded peptides (PCT Publication No. WO 93/20242); random bio-oligomers (PCT Publication No. WO 92/00091); diversomers, which are nonpeptide nonoligomeric compounds (De Witt et al., 1993, Proc. Nat. Acad. Sci. USA 90:6909-6913); vinylogous polypeptides (Hagihara et al., 1992, J. Amer. Chem. Soc. 114:6568); nonpeptidal peptidomimetics with a beta-D-glucose scaffolding (Hirschmann et al., 1992, J. Amer. Chem. Soc. 114:9217-9218); small organic compounds (Chen et al., 1994, J. Amer. Chem. Soc. 116:2661; Gordon et al., 1994, J. Med. Chem. 37:1385-1401); oligocarbamates (Cho et al., 1993, Science 261:1303); peptidyl phosphonates (Campbell et al., 1994, J. Org. Chem. 59:658); peptide nucleic acids (U.S. Pat. No. 5,539,083); antibody libraries (Vaughn et al., 1996, Nature Biotechnology, 14(3):309-314); carbohydrates (Liang et al., 1996, Science 274:1520-1522; U.S. Pat. No. 5,593,853); benzodiazepines (Bunin et al., 1994, Proc. Nat. Acad. Sci. USA 91(11):4708-4712; benzodiazepines U.S. Pat. No. 5,288,514); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337), and the like. In addition, numerous combinatorial libraries are themselves commercially available (e.g., ComGenex, Princeton, N.J.; Asinex, Moscow, Russia: Tripos, Inc., St. Louis, Mo.; ChemStar, Ltd, Moscow, Russia; 3D Pharmaceuticals, Exton, Pa.; Martek Biosciences, Columbia, Md., etc.).

Libraries of compounds may be presented in solution (Houghten, 1992, Biotechniques 13:412-421); on beads (Lam, 1991, Nature 354:82-84); chips (Fodor, 1993, Nature 364:555-556); bacteria (U.S. Pat. No. 5,223,409); spores (U.S. Pat. No. 5,223,409); plasmids (Cull et al., 1992, Proc Natl Acad Sci USA 89:1865-1869); or phage (Scott and Smith, 1990, Science 249:386-390; Devlin, 1990, Science 249:404-406; Cwirla et al., 1990, Proc. Natl. Acad. Sci. 87:6378-6382; Felici, 1991, J. Mol. Biol. 222:301-310). Each of the above-cited publications, patents, and patent applications is hereby incorporated by reference in its entirety.

In preferred embodiments, the test agent may be a small organic molecule. The small organic molecule may be a single synthetic or natural molecule. Alternatively, the small organic molecule may be a member of a combinatorial library of small organic molecules. The small organic molecule may range in size from about 10 Daltons (Da) to about 10,000 Da, more preferably from about 50 Da to about 5000 Da, and most preferably from about 100 Da to about 2000 Da.

In one embodiment, the test agent may be a phosphatidylinositol analog that is unable to be phosphorylated at position 3. Examples of suitable phosphatidylinositol analogs include, but are not limited to, phosphatidylinositol ether lipid analogs (PIAs) such as PIA-5, PIA-6, PIA-23, PIA-24, and PIA-25 (Kozikowski, et al., 2003, J. Am. Chem. Soc. 125:1144-1145), 1_{D}-3,4-dideoxyphosphatidylinositol ether lipid (Hu et al., 2000, Tetrahedron Let. 41(39):7415-7418), and _{D}-2,3-dideoxy-myoinositol 1-[(R)-2-methoxy-3-(octadecyloxy)propyl hydrogen phosphate] (SH-6; Alexis Biochemicals, San Diego, Calif.),

In another embodiment, the test agent may be an inhibitor of phosphoinositol-3 kinase (PI3K). Suitable PI3K inhibitors include demethoxyviriden, wortmannin, LY294002 (Vlahos et al., 1994, J. Biol. Chem. 269:5241-5248), minocycline (Yao et al., 2004, Circ. Res. 95(4):354-371), PIK-90 (Knight et al., 2006, Cell 125(4):733-747), SF1126 (Semafore Pharmaceuticals Inc, Indianapolis, Ind.), XL147 (Exelixis Inc., So. San Francisco, Calif.), ZSTK474 (Yaguchi et al., 2006, JNCI J. Natl. Cancer Inst. 98(8):545-556), IC486068 and IC87114 (ICOS Corporation, Bothell, Wash.).

In still another embodiment, the test agent may be an inhibitor of 3-phosphoinositide-dependent protein kinase 1 (PDK-1). Suitable PDK-1 inhibitors include 7-hydroxystaurosporine (also known as UCN-01), BX-320 (Berlex Biosciences, Richmond, Calif.), and OSU-03012 (a celecoxib derivative; Arno Therapeutics Inc., Fairfield, N.J.).

In yet another embodiment, the test agent may be an inhibitor of serum/glucocorticoid kinase-1 (SGK-1). Suitable SGK-1 inhibitors include indirubin-3′-oxime (i.e., 3-[1,3-dihydro-3-(hydroxyimino)-2H-indol-2-ylidene]-1,3-dihydro-2H-indol-2-one) (Hoessel, et al., 1999, Nat. Cell Biol. 1(1):60-67), and H-89 (i.e., N-[2-(p-bromocinnamylamino)ethyl]-5-isoquinolinesulfonamide. 2HCl) (Chijiwa et al., 1990, J. Biol. Chem. 265(9):5267-72).

In another alternate embodiment, the test agent may be an inhibitor of fatty acid and lipid-binding protein (LBP-6) or an inhibitor of F53H1.3, an unknown, highly conserved protein (see Example 6).

The test nematode may be contacted with a single test agent. Alternatively, the test nematode may be contacted with two or more test agents simultaneously. If the plurality of test agents modulates the stress response of the test nematode, then each of the agents in the plurality of agents may be screened individually to identify the agent (or agents) that modulates resistance to oxidative and/or electrophilic stress.

d. Screening Protocol

The nematodes may be placed in individual dishes, in 24-well plates, 96-well plates, 256-well plates, and so forth.

The temperature at which the screening method is performed may range from about 10° C. to about 30° C., preferably from about 15° C. to about 25° C., and more preferably from about 18° C. to about 22° C..

Contact of the test nematode with the test agent may precede exposure of the test nematode, the negative control, and the mutant nematode to the oxidative and/or electrophilic stress. The time interval between contact with the test agent and exposure to the oxidative and/or electrophilic stress can and will vary depending upon the agent being screened. The time interval may range from about 1 minute to about one or more days, and more preferably from about 5 minutes to about 60 minutes. Alternatively, contact with the test agent and exposure to the oxidative and/or electrophilic stress may occur simultaneously.

e. Stress Response

The stress responses of the test nematode, the negative control nematode, and the mutant nematode will be measured at an appropriate interval of time after exposure to the oxidative and/or electrophilic stress. The time at which the response is measured can and will vary depending upon the agent being screened and the response being measured. The response may be measured as early as about 1 minute after exposure to the oxidative and/or electrophilic stress or as late as about seven days after exposure to the oxidative and/or electrophilic stress. Preferably, the response is measured from about 1 hour to about 24 hours after exposure to the oxidative and/or electrophilic stress, and more preferably from about 2 hours to about 16 hours after exposure to the oxidative and/or electrophilic stress. The assay may be conducted as a time course at one or a few doses, or a dose-response series at one or a few assay times.

The stress response of the nematodes may be measured in vivo. In one embodiment, the in vivo assay may be short-term survival. Survival may be monitored by direct observation, i.e., by spontaneous movement of the nematode or by touch-provoked movement of the animal. Alternatively, survival may be monitored by optical detection of the nematodes and their movements. For example, an instrument may be used to determine optical density across the test surface, whereby animals may be detected by changes in density at the location where they were located. As another example, the animals may express a reporter gene (e.g., GFP) that is detectable in living animals, whereby a machine could monitor the animals using a suitable reporter gene detection protocol, e.g., fluorescence detection. In another embodiment, the in vivo assay may be a readily observable behavior of the nematode. In still another embodiment, the in vivo assay may be lifespan. Nematode lifespan assays are well known in the art (e.g., see the examples).

Alternatively, the response of the nematode may be measured in vitro. For this, the appropriate biological material (i.e., cell lysate, protein, nucleic acid, etc.) will be isolated from the animal at an appropriate period of time following exposure to the oxidative and/or electrophilic stress. In one embodiment, the in vitro assay may be the measurement of an enzyme activity. For example, the enzyme activity measured may be the total phosphorylation activity (e.g., see Example 4). Alternatively, the enzyme activity measured may be total protein kinase activity, total lipid kinase activity, or total phosphatase activity. Protein kinase activity may be measured using endogenous protein or using synthetic peptides as substrates. Alternatively, the enzyme assay may measure the activity of a specific enzyme, such as phosphatidylinositol 3-kinase (PI3K), AKT kinases, DAF-18/PIP3 phosphatase, superoxide dismutase, catalase, glutathione peroxidase, and so forth. A variety of enzyme assays are known in the art or are commercially available as kits.

In another embodiment, the in vitro assay may be the measurement of protein levels, whereby a protein profile may be generated. For example, the levels of phosphoproteins may be determined, and a phosphoprotein profile may be generated. Western blotting, ELISA analysis, immunolocalization, or another technique well known in the art may be used to assay proteins, such as those listed above. In still another embodiment, the in vitro assay may be the measurement of transcript levels, whereby a transcript profile may be generated. Transcripts coding proteins of interest may be detected using reverse transcriptase PCR, quantitative PCR, Northern blotting, or any of the other techniques known to those skilled in the art. In another alternate embodiment, the in vitro assay may be the measurement of metabolite levels, whereby a metabolite profile may be generated. For example, the metabolites may be a phosphatidylinositol molecule, such as PI(4,5)P2, PI(3,4,5)P3, and so forth. The methodology used to measure the metabolite can and will vary depending upon the nature of the molecules. In a further embodiment, the in vitro assay may be the measurement of lipid levels, whereby a lipid profile may be generated. In yet another embodiment, the in vitro assay may encompass microarray technologies. For this, commercially available arrays of nucleic acids, peptides, proteins, antibodies, lipids, or small molecules may be screened.

In a preferred embodiment, the stress response to be measured is survival. In this embodiment, survival is measured by direct observation from about 2 hours to about 16 hours after exposure to the oxidative and/or electrophilic stress.

The stress responses of the test nematode, the negative control nematode, and the mutant nematode are compared to determine whether the agent modulates the response to oxidative and/or electrophilic stress. The stress response of the mutant nematode, which has an extended lifespan and increased resistance to oxidative and/or electrophilic stress, differs markedly from that of the negative control nematode.

A change in the stress response of the test nematode away from that of the negative control nematode and towards that of the mutant indicates that the agent increases resistance to oxidative and/or electrophilic stress. An agent that changes the magnitude of the stress response of the test nematode to be similar to that of the mutant nematode indicates that the agent may provide excellent resistance to oxidative and/or electrophilic stress and extend lifespan. Agents identified as positive modulators of lifespan and/or oxidative and/or electrophilic stress may be subjected to additional tests. The biochemical pathway or specific protein affected by the agent may also be revealed via additional tests.

An agent that changes the stress response of the test nematode away from the responses of both the negative control and the mutant nematodes is an agent that decreases resistance to oxidative and/or electrophilic stress or induces oxidative and/or electrophilic stress itself. Such an agent may be useful as an anti-nematode agent, and also may reveal pathways and modes of intervention to have a beneficial effect on survival.

An agent that does not change the response of the test nematode relative to the negative control nematode does not affect resistance to stress.

Differences in the stress responses of the test and negative control nematodes will generally be statistically significant (e.g. at the 90%, 95%, or 99% confidence level), as determined using any statistical test suited for the data set provided, e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.).

### II. Methods for Identifying a Biomarker of Longevity or a Pathway Governing Longevity in Response to PIP3 Signaling.

The invention also provides methods for identifying biomarkers of longevity in the PIP3 signaling pathway and/or identifying pathways that govern longevity in response to PIP3 signaling. These methods take advantage of the phenotypic differences between the long-lived age-1 null nematodes and wild type or other age-1 mutants to tease apart the molecules and the PIP3 signaling pathways that confer the greatly extended lifespan and increased stress resistance to the long-lived age-1 null nematodes. Identification of longevity biomarkers and/or PIP3 signaling pathways may lead to the development of therapeutic interventions for treating premature aging diseases, for treating diseases or conditions that are part of the normal aging process, for slowing the aging process or extending lifespan, and for treating diseases or conditions causes by oxidative and/or electrophilic stresses.

a. Altering PIP3 Signaling in a Wild Type Nematode

A method is provided that comprises contacting a nematode with a treatment that increases lifespan and resistance to oxidative and/or electrophilic stress. The method comprises contacting a wild type nematode with at least one treatment that reduces the levels of PIP3 or alters the function of PIP3. Typically, PIP3 interacts with proteins by binding to a pleckstrin homology (PH) domain within the PIP3 interacting protein. Binding of PIP3 generally leads to either a cellular translocation of the protein (e.g., to the plasma membrane) or an allosteric conformational change in the protein. Some of the proteins that interact with PIP3 have kinase activity (e.g., PDK-1, AKT-1, AKT-2, etc.), whereby these kinases phosphorylate and regulate the activity of downstream proteins. Among the PIP3 interacting proteins are PIP3 phosphatases (one of which is also known as PTEN), which are enzymes that convert PIP3 into PIP2. Non-limiting examples of PIP3 interacting proteins, therefore, include PI3K, PDK-1, AKT-1, AKT-2, SGK, SMK-1, and PIP3 phosphatases (including PTEN).

In one embodiment, the treatment may comprise overexpressing a PIP3 interacting protein. Methods for overexpressing a protein are well known in the art (for reference, see Ausubel et al. (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y.). For example, a nucleic acid construct encoding a PIP3 interacting protein may be introduced into the test nematode. The nucleic acid encoding the PIP3 interacting protein may be under the control of a strong constitutive promoter or a tissue specific promoter. The nucleic acid construct may be introduced into a chromosome of the nematode or the nucleic acid construct may be introduced extrachromosomally. In an alternate aspect of this embodiment, a treatment may comprise overexpression a PH domain or overexpressing a polypeptide comprising one or more PH domains or part of such domains. Furthermore, such domains may be introduced into cells, tissue or whole animals (including humans) that do not express them. Methods for inducing uptake of polypeptides are known in the art, and include (but are not limited to) addition of extensions comprising predominantly basic amino acids (e.g., lysine, arginine) to either terminus of the polypeptide; or fusion with the HIV tat protein or other viral proteins mediating viral entry into host cells.

In another embodiment, the treatment may comprise blocking expression of a PIP3 interacting protein. Methods for blocking gene expression or protein expression are known to those of skill in the art (see references listed above). As an example, the gene coding the PIP3 interacting protein or a control region of the gene coding the PIP3 interacting protein may be inactivated by mutation or deletion. Alternatively, antisense or RNA interference technology may be used to block expression of a PIP3 interacting protein, either transiently or by stable integration.

In still another embodiment, the treatment may comprise expressing a PIP3 interacting protein that has an altered response to PIP3. Modifying the PH domain of a PIP3 interacting protein may alter the response to PIP3. For example, the PH domain may be modified such that it is active in the absence of PIP3. Alternatively, the PH domain may be modified such that it cannot be activated by PIP3. Likewise, if the PIP3 interacting protein is a kinase, then its kinase domain may be altered such that it is constitutively active. Or the kinase domain may be modified such that it has no kinase activity, and so forth. Similarly, a phosphorylation site on a PIP3 interacting protein may be modified such that it cannot be phosphorylated, or that it cannot be dephosphorylated, etc. One skilled in the art will know how to achieve the desired modification, as well as know how to introduce the nucleic acid encoding the altered PIP3 interacting protein, or the protein or polypeptide itself, into the recipient cells or organism. Included in the above embodiment is the possibility of modifying a PH domain in such a way as to increase its binding to PIP3, or cause it to fail to release PIP3 once bound.

In yet another embodiment, the treatment may comprise overexpressing a phosphatidylinositol (4,5) diphosphate (PIP2) binding protein, which may lead to reduced levels of PIP3. The PIP2-binding domain may be itself overexpressed, or modfied in such a way as to increase its binding to PIP2, or cause it to fail to release PIP2 once bound. Any such domain may be overexpressed via introduction of a corresponding DNA expression vector, or introduced directly, as described above.

After the levels of PIP3 have been reduced or the function of PIP3 has been altered in the treated nematode, the treated nematode and an untreated control nematode may be exposed to an oxidative and/or electrophilic stress, as detailed above in section I(b) and (e). The stress responses of the two nematodes are compared to determine whether the treatment modified resistance to the oxidative and/or electrophilic stress. In a preferred embodiment, the stress response is short-term survival. A treatment that increases the survival time of the treated nematode relative to the negative control nematode indicates that the treatment altered a longevity biomarker or a PIP3 signaling pathway that mediates oxidative and/or electrophilic stress responses and longevity.

The method may be modified such that resistance to an oxidative and/or electrophilic stress may not be evaluated, but rather another parameter may be assessed. For example, the lifespan of the treated and untreated nematodes may be compared. Alternatively, differences in developmental timing through the larval stages of development may be compared between the treated and untreated nematodes. Similarly, differences in fertility and/or fecundity may be compared. In another embodiment, a visible output, such as expression of a fluorescent protein, may provide a readout indicative of the efficacy of the treatment in modulating oxidative and/or electrophilic stress. A positive result in a short-term assay may lead to further testing of long-term responses such as life span.

b. Altering PIP3 Signaling in a First Generation Age-1 Null Mutant or an Age-1(hx546) Mutant

Also provided is a method that comprises contacting a first generation age-1 null mutant C. elegans or a weaker age-1 mutant allele (e.g., hx546) of C. elegans with a treatment that further increases lifespan and/or resistance to oxidative and/or electrophilic stress. The method comprises contacting the age-1 mutant with at least one treatment that reduces the levels of PIP3 or alters the function of PIP3. The possible treatments were detailed above in section II(a). The stress response (or another response, as detailed above) of the treated age-1 mutant may then be compared to the response of the extremely long-lived, second generation age-1 null C. elegans. This method seeks to identify the mechanisms that lead to the greatly extended lifespan of the second-generation age-1 null mutants relative to the first generation age-1 null mutants or age-1 mutants with weaker alleles. A change in the stress response of the treated age-1 mutant to be more similar to that of the second generation age-1 null nematode indicates that the treatment identified a longevity marker or a PIP3 signaling pathway that mediates stress responses and longevity.

c. Reversing the Phenotype of the Extremely Long-Lived Age-1 Null Nematode

Lastly, a method is provided that comprises contacting a long-lived second generation age-1 null nematode with a treatment that reverses its extended lifespan and reverses its increased resistance to oxidative and/or electrophilic stress, thereby leading to discovery of downstream components of the signaling pathway that leads to these traits. Age-1 null mutants essentially lack PI3K, and by the second generation have no or extremely low levels of PIP3. Thus, second generation age-1 null mutants essentially lack any activated PIP3 interacting proteins. The method comprises contacting a second-generation age-1 null nematode with at least one constitutively active PIP3 interacting protein. PIP3 interacting proteins were described above section II(a). The PIP3 interacting protein may be made constitutively active by altering the kinase domain, altering a phosphorylation site, or altering the PH domain. One skilled in the art will know how to modify the nucleic acid encoding the PIP3 interacting protein and how to determine whether the modified protein is constitutively active. One of skill in the art will also know how to introduce the nucleic acid encoding the constitutively active PIP3 interacting protein into a second-generation age-1 null nematode.

The stress response (or another response, as detailed above) of the treated second-generation age-1 null mutant is then compared to the response of an untreated second-generation age-1 null mutant C. elegans. In a preferred embodiment, the stress response is short-term survival. Thus, if the treated second-generation age-1 null nematode has a shorter survival time than the untreated second-generation age-1 mutant, then the modified PIP3 interacting protein may play an important role in the longevity signaling pathway or pathways. The nature of the modification to make the PIP3 interacting protein constitutively active may help reveal the mechanism by which the PIP3 interacting protein normally responds to PIP3 signaling. That is, whether the response is due to the docking of the PIP3 interacting protein to a particular cellular location or whether it is due to an allosteric conformational change in the PIP3 interacting protein.

d. Therapeutic Interventions

The methods provided in this invention may lead to the identification of agents that may be used therapeutically to treat premature aging diseases, to treat diseases or conditions caused by oxidative and/or electrophilic stresses, and to extend lifespan. Non-limiting examples of premature aging diseases include Werner syndrome, Hutchinson-Gilford disease, Bloom syndrome, Cockayne syndrome, ataxia telangiectasia, and Down syndrome. Many types of diabetes, such as nearly all type-2 diabetes, some type-1 diabetes, and insulin resistant diabetes, may lead to premature aging. Diseases involving oxidative and/or electrophilic stress include neuronal ischemia during stroke, post-cardiopulmonary bypass syndrome, brain trauma, and status epilepticus; cardiac damage induced during ischemic heart disease; renal damage induced by ischemia and toxins; chronic neurodegenerative disorders, such Parkinson's disease, amyloidoses, prion disorders, and Alzheimer's disease; and autoimmune diseases. Nondisease examples include ischemia-reperfusion injury suffered in the course of surgical intervention for trauma or disease.

### DEFINITIONS

The term “maternal carryover,” as used herein, refers to components present in the oocyte that once fertilized will produce an embryo. The components include silent (i.e., untranslated) RNA molecules, storage proteins, as well as other maternal RNAs, proteins, and small metabolites.

As used herein, the terms “response” or “stress response” refer to a physiological response or a molecular response to an oxidative and/or electrophilic stress.

The term “second generation,” as used herein, refers to the second generation in which the indicated (recessive) mutation exists in a homozygous state or to a later generation of homozygous mutant nematode in which fertility has been partially restored.

### EXAMPLES

The following examples illustrate various embodiments of the invention.

### Example 1

### Ten-Fold Increased Lifespan of Age-1 Null Mutant Nematodes

To date, the greatest extensions of longevity by single mutations have been about 2.6-fold in C. elegans, nearly two-fold in Drosophila, and only about 1.3- to 1.5-fold in mice. It is clear, however, that these mutants do not define the upper limit for life extension, since combinations of two or three interventions, e.g. augmenting such mutations with germ-cell ablation or dietary restriction, can result in life extension by as much as 6-fold in the nematode. Similarly, the greatest life extension obtained thus far in mice, 1.7-fold, required the combined effects of a life-extending mutation and caloric restriction. Many long-lived mutations also confer resistance to a variety of stresses, whereas naturally occurring genetic variants, known as longevity QTLs, display selective resistance to some stresses and not others.

Nematode strains were obtained from the Caenorhabditis Genetics Center (CGC; Minneapolis, Minn.) or were derived from CGC-supplied strains. Working stocks were initiated as needed (typically every 3 months) from aliquots stored frozen at −80° C. or under liquid nitrogen. Unless noted otherwise, worm strains were maintained at 20° C. on NG agar plates supplemented to 1% with Bacto-Peptone and spread with a lawn of E. coli strain OP50, as described previously (Sulston and Hodgkin, in The nematode Caenorhabditis elegans (1988) W. B. Wood (Ed.) Cold Spring Harbor Laboratory Press, Cold Spring Harbor, N.Y., pp. 587-606; Ebert et al. (1993) Genetics 135:1003; Ayyadevara et al. (2003) Genetics 163:557).

Three alleles of age-1, i.e., strains TJ1052 [age-1(hx546)], GR1168 [age-1(mg44)/mnC1], and DR722 [age-1(m333)/mnC1], were obtained. Each allele was outcrossed six generations into N2DRM (CGC “N2 Male Strain”), a direct continuation of the Bristol-N2DRM stock, to eliminate allelic differences at other loci (“background effects”). Hx546 is a reduction-of-function point mutation, while the other two alleles are nonsense mutations in which stop codons truncate the PI3K protein before the kinase active site. DNA sequence of age-1 exons confirmed that the TGA/amber stop codon replacing TGG (Trp) at amino acid 387 in age-1(mg44) worms (strain SR808 after outcrossing into N2DRM) corresponded to the mutation described previously at position 405 (not shown). Similarly, it was confirmed that the age-1(m333) mutation (strain SR809 after N2DRM outcrossing) as a TGG (Trp) to TAG/opal change at position 641, corresponded to residue 659 in the previous sequence (not shown). Although amino acid numbering has been modified over the last decade, with the inclusion of alternative exons, the mutations and their flanking sequences were identical to those reported (Morris et al. (1996) Nature 382:536).

Worms were grown on NG plates with 0.6% peptone and harvested by rinsing with S buffer (0.1 M NaCl, 0.05 M potassium phosphate, pH 6.0). Adult, >99.6% hermaphrodites, were allowed to settle and then were suspended in alkaline hypochlorite (5 min at 20° C. in 0.5 N NaOH, 1.05% hypochlorite). Uneclosed larvae (“eggs”) were recovered, rinsed with S Buffer, and transferred to fresh agar plates containing E. coli OP50. Survival cultures (in 60-mm dishes) were set up one day after the L4/adult molt by transferring 25-50 adults to 60-mm dishes containing, for solid and liquid survivals respectively, NG agar with an OP50 lawn, or 3 ml of Survival Medium (S Buffer containing 10 μg/ml cholesterol and 2×109 OP50 cells/ml). Worms were incubated at 20° C. unless otherwise noted; live worms were counted and transferred daily to fresh dishes prepared as above. Those failing to move spontaneously or in response to touch were counted as dead. Worms lost, stranded (e.g., on dish walls or beneath the agar) or ‘bagged’ (endotokia matricida) were scored as ‘censored’ at the midpoint of the measurement interval in which the event occurred; those inadvertently killed were censored at the time of the event. Survival times were compared by rank ordered nonparametric tests and t-tests.

The two age-1 null mutant strains, producing truncated AGE-1 proteins, were maintained as heterozygotes with the mnC1 balancer chromosome. Of the progeny arising from self-fertilization of these heterozygotes, one-fourth was homozygous for the age-1 mutation. The essentially normal development of such age-1/age-1 homozygotes was attributed to maternal protection due to wild type age-1 mRNA or AGE-1 protein encoded by the normal age-1 allele on mnC1. Their progeny that lacked any maternally-contributed AGE-1 or its PIP3 product, however, constitutively formed dauer larvae at 25.5° C., as reported previously (Larsen et al. (1995) Genetics 139:1567; Tissenbaum and Ruvkun (1998) Genetics 148:703). When the progeny were grown at 15° or 20° C., however, 95% of mg44/mg44 progeny of mg44/mg44 hermaphrodites matured to adults within 8-10 days (FIG. 1, A and B; Table 1). Development was even more protracted for progeny of maternally-protected m333/m333 homozygotes. Only 2-3% formed nearly full-sized adults within 16-19 days, and another 5-10% formed abnormally small adults (“runts”).

TABLE 1
Development times and adult survival times for wild type and age-1 mutant C. elegans.

| | Developmental | Developmental | Developmental | | |
|---|---|---|---|---|---|
| Strain, | time, 15 ± 0.1° C. | time, 20 ± 0.1° C. | time, 25.5 ± 0.1° C. | Median survival at | Median survival** |
| outcross | (% adult | (% adult | (% adult | 20° C. | relative to |
| generations | formation, N) | formation, N) | formation, N) | (adult days) | N2DRM |
| N2DRM (control) | 3.5 d (100%, 200) | 2.5 d (99%, 200) | 2.0 d (98%, 200) | 14.6, 16.0, 16.5 | 1.00 ± 0.06 |
| age-1(hx546), x6 | 4.5 d (99.5%, 203) | 3 d (98.5%, 198) | 2.5 d (94%, 207) | 30.5, 33.5 | 2.03 ± 0.13^{a} |
| age-1(m333), x6 | 19 d (3%, 207) | 16 d (2%, 207) | Indefinite (0%, 189) | 160 | 10.2 |
| age-1(mg44), x6 | 10 d (95%, 207) | 8 d (94.7% ± 1.5*, 621) | Indefinite (0%, 540) | 120, 160, 184 | 9.9 ± 2.0^{b} |
| age-1(mg44), x0 | 10 d (N.D.) | 8 d (N.D.) | Indefinite (N.D.) | 149, 160, 193 | 10.7 ± 1.5^{b} |

Note:
N2DRM and age-1(hx546) worms were propagated as homozygous lines. Other age-1 homozygotes arose by self-fertilization of age-1(m333)/mnC1 or age-1(mg44)/mnC1 heterozygotes, and their progeny (“second-generation” age-1 homozygotes) were tested as indicated. Developmental time is taken as the age at 90% of maximal adult formation; percent adult formation (in parentheses, followed by the number of worms counted) was scored 1-2 days later as the number of full-size adults. Age-1(m333) produced ~10% of adult “runts” at 15° C., or ~5% at 20° C., which were excluded from survivals.
N.D., not determined.
*The mean ± SD is shown for 4 independent groups totaling 621 worms.
**The mean of 2 or 3 median survival times, ±1 SD, divided by the N2DRM mean.
^{a}Different from N2DRM, t-test P < 0.02;
^{b}Different from N2DRM or age-1(hx546), t-test P < 0.01.

Second-generation homozygous age-1(mg44) adults appeared normal apart from a slight protrusion of the vulva (FIG. 1B, arrows), and had near-normal motility (not shown) and pharyngeal pumping: 151 beats/min vs. 154/min for N2DRM and 155/min for age-1(hx546), each at day 1 of adulthood. Like wild type adults but unlike dauer larvae, they were fully susceptible to disruption by 1% sodium dodecyl sulfate (10/10 killed). In contrast, age-1(m333) adults were smaller than normal and had slightly reduced motility and pharyngeal pumping rate (128/min, 83% of the N2DRM rate), perhaps due to other linked mutations. However, adult worms bearing either homozygous m333 or mg44 nonsense mutations (and born to age-1 /age-1 hermaphrodites, hence lacking any maternal protection) were much longer lived than any mutant worms previously reported. The lifespan of these mutant worms averaged 10 times than the median or 90^{th} percentile adult lifespan of the congenic N2DRM controls (FIG. 1C and Table 1). This was not due to background gene effects, i.e. stock-specific genetic interactions with other loci, since the original mutant stocks survived just as long as those outcrossed six generations into the N2DRM background (FIG. 1C). In common with some of the longevitous groups produced by multiple interventions, most age-1 null worms that survived beyond the population median lifespan (145-189 adult days, FIG. 1C) showed vigorous spontaneous movement until the last 1-2 weeks of life.

The increase in total lifespan, due to both developmental and adult lifespan effects of the age-1(mg44) nonsense mutation (Table 1), were largely or entirely reversed by a second mutation, daf-16(m26), which inactivated the DAF-16/FOXO forkhead transcription factor downstream of the AGE-1 PI3-kinase (FIG. 2, A and B).

### Example 2

### Exceptional Stress Resistance of Age-1 Null Mutants

To determine whether these extreme-longevity strains were also unusually resistant to stresses, day-2 adults were subjected to variety of stresses. First, thermal tolerance was tested by shifting the temperature from 20° to 35.5° C.. None of the three age-1 mutant alleles tested were more tolerant of high temperature than wild type worms (not shown). Next, resistance to two oxidative stresses, and resistance to an electrophilic stress associated with lipid oxidation were tested. For this, young adult worms were transferred to wells of a 24-well plate (25 worms/well) at 9 days post-hatch for second-generation age-1(mg44) homozygotes derived from strains SR808 and GR1168; at 19 days for second-generation age-1(m333) homozygotes derived from strains SR809 and DR722; and at 5 days for all other strains. Worms were maintained at 20° C., in S Medium (S Buffer plus 0.5% cholesterol) also containing 3 or 5 mM hydrogen peroxide, 10 mM 4-hydroxynonenol (4-HNE), or 150 mM paraquat. Worms were scored for survival, as described above, at regular intervals—typically once per hour.

Short-term survival in the presence of these stress-inducing agents is presented in FIG. 3. In the presence of hydrogen peroxide, the two very-long-lived age-1 alleles survived roughly 9- and 8-fold longer than wild type in 3 and 5 mM H₂O₂, respectively (FIG. 3, A and B) (i.e., mortality at 30 hr for those alleles, was similar to that of wild type at 3.3 or 3.8 hr). Survival in 150-mM paraquat, which generates superoxide, was also extended by 3- to 5-fold (FIG. 3C). Electrophilic toxicity of 4-HNE, a lipoperoxidation end product, was postponed at least 4-fold (FIG. 3D). In each case, stress resistance was markedly greater for age-1(mg44) and age-1(m333) worms than those bearing the weaker age-1 allele, hx546.

A second mutation, daf-16(m26), disabling the forkhead transcription factor believed to mediate insulin-like signaling in C. elegans, was also assessed for its ability to epistatically reverse the resistance to 4-HNE and paraquat. The daf-16(m26) mutation fully reverted the stress-resistance trait of the weaker age-1 allele (hx546), but only partially reversed protection due to the stronger mg44 and m333 alleles: 80% for 4-HNE, but just 40% for paraquat (FIG. 3, C and D). Although this result appeared to be at odds with the full reversion, by the same daf16(m26) mutation, of lifespan effects arising from all mutant alleles of age-1, in fact age-1 (hx546);daf-16 double mutants survived significantly less time in liquid medium than age-1 (mg44);daf-16 or wild type controls (FIG. 2B), in keeping with previous reports that daf-16 defective strains are 15-25% shorter lived than their normal counterparts. Relative to age-1(hx546);daf-16, taken as the “baseline” for survival of a daf-16 mutant strain, longevity reversion of age-1(mg44) by daf-16 would also be considered incomplete.

### Example 3

### A PI3K Requirement for Oogenesis

Homozygous-null mutants of age-1 were fertile when maternally protected, yielding 150±14 (SEM) progeny per SR808 (mg44) hermaphrodite, and 131±13 progeny per SR809 (m333) parent. From >1000 of those offspring, born of age-1 null parents, no progeny were produced (differing from their parents, each P<0.005), and neither embryos nor unfertilized oocytes were laid. To further examine the role of age-1 during oogenesis, worms were stained with a nuclear DNA dye so that germ cell nuclei could be visualized. For this, worms were washed and fixed according to the protocol of Finney and Ruvkun (Cell (1990) 63:895). DAPI (4′,6-diamidino-2-phenylindole) was added to PBS (phosphate-buffered saline, pH 7.0) to a final concentration of 0.1 μg/ml, and worms were incubated for 30 min at room temperature with this mixture. Worms were then washed 3× with PBS and GIF images were captured on a Nikon Cool-Pix digital camera through a Nikon Eclipse E1000 fluorescence microscope.

DAPI-stained nuclei of day-1 adults (FIG. 4, A-C) confirmed the presence of germ-cell syncytia in second generation mg44 homozygous nematodes (arrows, panel B) that contained far fewer nuclei than their wild type (panel A) or age-1(hx546) (C) counterparts. In the total absence of full-length PI3K, these syncytial nuclei failed to complete oogenesis, and produced no mature oocytes or embryos (FIG. 4, compare panel B to A and C). Because spermatozoa also appeared to be deficient, age-1(mg44) hermaphrodites were mated with N2DRM males to determine whether any mature oocytes were formed. Again, no progeny were observed, confirming that oocyte development was fully blocked in worms that lacked endogenous or maternally-provided phosphatidylinositol-3-kinase.

### Example 4

### Decreased Protein Phosphorylation in Age-1 Null Mutants

The AGE-1 protein is a class I catalytic subunit of phosphatidylinositol-3-kinase (PI3K_{CS-I}), which converts phosphatidylinositol-4,5-phosphate to phosphatidylinositol-3,4,5-phosphate, PI(3,4,5)P3. PI3K also has protein kinase activity, as evidenced by phosphorylation of its own regulatory subunit. PI(3,4,5)P3, however, is regarded as the principal effector through which PI3K modulates other kinases, including AKT-1, AKT-2 and PDK-1. Furthermore, PI3K is a docking factor for many membrane-associated kinases, as well as an allosteric activator of AKT-1. Although phosphatidylinositol di- and tri-phosphates can be measured directly, the thousand-fold higher levels of PIP2 obscure the normal or diminished levels of PIP3. Lacking a sensitive direct assay of PIP3, the levels of protein phosphorylation in vitro were instead assessed to provide a functional test for attenuation of multiple kinase activities, the predicted consequence of PIP3 depletion.

Worms grown at 20° C. were collected and quickly frozen in liquid nitrogen to preserve the activity of endogenous kinases. Lysates were prepared in the presence of 50-mM Tris pH 7.5, 80-mM β-mercaptoethanol, 2-mM EDTA and 1-mM PMSF, supplemented with Protease Inhibitor Cocktail (CalBiochem; San Diego, Calif.). Worms were ground over dry ice, by mortar and pestle, and then sonicated on ice (VIRTIS Virsonic 475, setting 2.5) in three 10-s pulses. Supernatants were collected following centrifugation (10 min, 11,000 g) to remove large organelles and membranes. In vitro phosphorylation was assessed with a constant load (20 or 30 μg, in two experiments) of protein per sample, in 100 μl of buffer containing 50-mM Tris pH 7.5, 12.5-mM MgCl₂ to which was added 8-10 μCi γ-³²P-ATP (NEN; Boston, Mass.). Phosphorylation reactions were incubated 1 min at 30° C., and then returned to an ice-water bath. Macromolecules were resolved by electrophoresis on 10% SDS-polyacrylamide gradient gels (Invitrogen; Carlsbad, Calif.), which were stained with SYPRO® Ruby (Invitrogen) to confirm constant protein loads, and dried under vacuum. ³²P β-emissions, from discrete bands migrating slower than a 25-kDa protein size marker (Benchmark Prestained Protein Ladder; Invitrogen), were quantitated for each lane after a 6-h exposure (Storm Phosphorimager and ImageQuant software; Molecular Dynamics, Sunnyvale, Calif.).

Lysates of day-5 adult worms supported much lower protein phosphorylation in vitro when derived from second-generation age-1(mg44) homozygous worms, than from wild type N2DRM worms (FIG. 5). Moreover, these mg44 worms displayed significantly less in vitro kinase activity than their genetically identical, but maternally protected parents, which were far less longevitous. A second mutation in the daf-16 locus did not reverse the broad suppression of protein kinase activity (not shown), as it cannot redress the knockout of PI3K. Since addition of a daf-16 mutation did largely revert the extreme-longevity effects of age-1 null mutations, it is concluded that PIP3 depletion, and consequent suppression of kinases other than PI3K, must require functional DAF-16 in order to produce hyper-longevity.

Next, in vitro kinase activity was compared among five age-1 mutant groups, each normalized to the wild-type strain N2DRM (FIG. 6, A-C). Panels A and B illustrate a typical experiment, and panel C summarizes results for replicate experiments with independent biological expansions of each group. The age-1(hx546) allele, showed 32% less kinase activity than N2DRM (FIG. 6C). Adults bearing the age-1(mg44) allele had total kinase activity <9% that of wild-type worms, whether maternally protected first-generation (F1) or very long-lived F2 homozygotes. There was only a marginally significant difference in kinase activity (P=0.05) between F1 and F2 worms: 7.3 vs. 8.6% of the N2DRM level, respectively (FIG. 6C). Eggs contained substantially less kinase activity than worms, per weight of protein, and thus could not augment the kinase activity of N2DRM worms; moreover, F1 adults had similar kinase levels whether gravid or post-gravid, and eggless dauer larvae had activity exceeding N2DRM adults (FIG. 7).

The consequences of adding a daf-16 mutation were quite distinct for the two age-1 alleles: for age-1(hx546) worms, the daf-16(m26) mutation more than doubled in vitro kinase activity, from 68% of wild-type to 160%, whereas the same daf-16 allele restored roughly half of the kinase deficiency resulting from the stronger age-1(mg44) mutation (FIG. 6C). Staining for total protein showed that loads scarcely differed among samples, despite variation in banding patterns (FIG. 6A). Insofar as kinase suppression was reversed in daf-16; age-1(mg44) double mutants, it was inferred that activity was partially inhibited through the DAF-16 ForkHead (FOXO) transcription factor; however, reversion by either the m26 or mu86 (deletion) allele of daf-16 was far from complete (see FIGS. 6C and 7), implying that at least half of observed kinase silencing was DAF-16-independent—perhaps reflecting direct effects of PIP3 depletion on kinases. This implicates pathways to increased longevity not previously explored.

In view of the diminished kinase activity of age-1(mg44) worms, it was anticipated that their protein phosphorylation would also be impaired. To detect in vivo phosphoproteins, total protein was extracted from each strain expansion, as described above. Protein (20 μg) was loaded onto NUPAGE® Novex 4-12% Bis-Tris gels (Invitrogen) and electrophoresed for 1 hour at 200V. Phosphoproteins were detected with Pro-Q Diamond stain (Invitrogen), for which fluorescence intensity is a linear function of protein concentration over a 1000-fold range, and also depends predictably on the number of phosphates per molecule. Total protein was assessed with Coomassie Blue gel stain (BioRad; Hercules, Calif.) to confirm equal load of proteins from each strain. PeppermintStick™ phosphoprotein molecular weight marker is composed of two phosphorylated (23.6 kDa and 45 kDa) and four unphosphorylated proteins (14.4 kDa, 18 kDa, 62.6 kDa and 116.25 kDa), which served as controls to assess specificity of phosphoprotein staining.

FIGS. 6D, E, and F show gels stained for phosphoproteins from wild-type and age-1 mutant strains of C. elegans. Total protein stain, shown in FIG. 6D, demonstrates even loading, while FIG. 6E shows the same gel restained with Pro-Q Diamond (Invitrogen) to detect and quantify phosphoproteins. Results for three replicates (using independent worm expansions for each strain) are summarized in FIG. 6F. Relative to wild-type N2DRM, age-1(hx546) worms had 16% less phosphoprotein staining (marginally significant at P<0.05), while F2 age-1(mg44) homozygous adults showed a 41% reduction in steady-state phosphoprotein level (P<0.001). The daf-16(m26) mutation restored either allele to ˜92% of the N2DRM level. This finding was also supported by 2-D dual-fluorphosphoprotein gels comparing F2 age-1(mg44) and N2DRM (FIG. 8). The observation of less deficiency for total phosphoprotein content than for protein kinase activity, in age-1(mg44)-homozygous F2 adults, may indicate that the severe (nearly 14-fold) decline in kinase activity can be partially offset by increased turnover of protein targets and/or their phosphate groups.

### Example 5

### Differential Gene Expression in Age-1 Null Mutants

The role of PI3K in the insulin/IGF-1 signaling (IIS) is to form PIP3—required for PDK-1 and SGK-1 activation of AKT kinase, which phosphorylates DAF-16. Because extreme age-1 mutants make no active PI3K (only truncated protein is seen on western gels), addition of a daf-16 mutation can restore activity only to kinases transcriptionally silenced by DAF-16, but not to kinases directly dependent on PIP3. The finding that daf-16 mutations restore nearly half of the mg44 kinase deficiency, and >70% of protein-phosphorylation deficit, implies that their suppression in age-1(mg44) is mediated in part by DAF-16. To test DAF-16 transcriptional silencing of kinase genes, the effects of age-1 alleles, with or without added inactivation of daf-16, were examined on transcript levels for IIS genes and a panel of other kinases and signaling components, including many thought to engage in cross-talk with the IIS pathway.

Gene expression levels of IIS pathway members and selected kinases were assessed by real-time, reverse-transcription PCR. Total RNA samples from each strain (post-gravid) were purified with RNeasy Mini kit (Qiagen, Germantown, Md.). cDNAs were reverse transcribed from total RNA preparations with SuperScript III First-Strand Synthesis System for RT-PCR (Invitrogen). Real-time PCR was conducted on an Opticon2 Thermal Cycler (MJ Research; Waltham, Mass.), using the SYBR® Green PCR Master Mix (Roche; Nutley, N.J. or Takara, Shiga, Japan).

As shown in FIG. 9, transcript levels were suppressed in age-1(mg44) F2 adults, for all tested components of the IIS pathway except akt-1. Relative to isogenic N2DRM controls, these F2 homozygotes showed reduced transcripts of the daf-2 gene encoding insulin/lGF-1 receptor (down 35×); age-1 itself, encoding the class-I catalytic subunit of PI3K (down 32×); the daf-18 (pten) phosphatase gene that opposes it (down 40×), and pdk-1, encoding 3-phosphoinositide-dependent kinase 1, which phosphorylates and activates the AKT-1 kinase required for IIS activation of DAF-16 (down 16×). Transcript levels were also assessed for two targets known to be induced by DAF-16, and thus serving as positive controls: sod-3 (up 15×), encoding mitochondrial superoxide dismutase-3, and R11A5.4 (up 12×), encoding phosphoenolpyruvate carboxykinase (PEPCK), an activator of gluconeogenesis. Moreover, similar although less striking suppression was observed for both F39B1.1, encoding a class-II PI3K_{CS} homolog (down 13×), and vps-34, encoding a class-III PI3K_{CS} subunit involved in vesicular trafficking and autophagy (down 4.4×). None of the observed transcript differences were significant for the weaker age-1 allele, hx546, and all were partially or completely reversed by a second mutation disabling the DAF16 transcription factor.

In view of the global suppression of kinase activity, the transcript levels of other kinases and signal-transduction genes were also examined. For this, the number of biological replicates was increased (to as many as 11 per group), and two additional groups were added to the panel: F1 homozygous age-1(mg44) adults and wild-type (N2DRM) dauer larvae (FIG. 8). Data for the weaker age-1(hx546) and daf-16(m26) alleles were similar to those presented in FIG. 9, and are presented in FIG. 11. The results of these extended surveys (FIGS. 10 and 11) confirm widespread transcriptional silencing of many signal-transduction genes in age-1(mg44) F2 adults, with marked and significant suppression (>3-fold relative to N2DRM, P<0.01) for 23 of the 35 genes tested. This silencing requires DAF-16, in that it is largely or entirely reversed by mutation to daf-16 (either the m26 or mu86 allele) in 23 of 23 genes. Not all DAF-16 targets were silenced; up-regulation was seen for two known positive targets of this transcription factor, sod-3 (FIG. 10L), and R11A5.4/pepck (FIG. 10N). Moreover, transcript levels of ins-1 (FIG. 10D), encoding an insulin-like peptide antagonist of IIS, and aak-2 (FIG. 10P), found to increase lifespan when over-expressed, are elevated in age-1(mg44) F2 adults and are higher still in dauer larvae. Transcriptional activation, like suppression, occurs via DAF-16.

Transcriptional modulation was also seen for first-generation (F1) age-1(mg44) adults, and was sometimes as great as for F2 adults (see FIG. 10A, C and G); however, expression of most genes was altered 2- to 8-fold more in F2 worms than in F1 (see FIG. 10I-P). F2 age-1(mg44) adults also showed far more pronounced effects than the weaker age-1 allele hx546 (FIGS. 9 and 11). Overall, transcript levels differed significantly between F2 and F1 generations of age-1(mg44) in 7 out of 16 signal-transduction genes surveyed (44%), illustrating that loss of maternal protection dramatically altered expression (FIG. 10). For most of the signal-transduction genes surveyed (9/16, 56%), age-1(mg44) F2 adults also differed significantly from dauer larvae (FIG. 10), indicating that these two long-lived populations, which differed with respect to developmental stage, morphology and behavior, also displayed quite distinct expression profiles.

Expression profiles of additional genes selected from those with the highest rankings based on SAM analyses of microarray data (i.e., full-genome microarrays comprising >22,000 synthetic oligomers probed with RNA preparations from multiple biological preparations of very long-lived age-1 alleles (mg44 and m333), the weaker age-1(hx546) allele with life extension more typical of long-lived IIS mutants, and N2DRM, a near-isogenic wild-type control strain) were generated via real-time PCR. FIG. 12 presents the main patterns of gene expression observed.

By far the most common pattern was “modulation proportional to longevity”; for 22 genes, transcripts were strongly and significantly suppressed (FIG. 12C, F-H, M-R, T, V and X) or activated (FIG. 12AE-AG) in age-1 (mg44) relative to N2DRM, but much less affected in age-1(hx546) adults, and still less (or not at all) in the daf-16 double mutants. Sixteen of the transcript-level differences between hx546 and N2DRM worms achieved nominal significance (P<0.01), well in excess of the single expected false positive, although only five met the strict Bonferroni criterion of P<0.0006. Thus, the great majority of these differences are likely to be real, although a few apparent instances of “modulation proportional to longevity” may actually belong to a second class of genes wherein transcripts were altered only for the kinase-null age-1(mg44) allele. The latter group is exemplified in FIG. 12E, U, W, Z-AD, AH, and AI. For two genes (FIG. 12B, S), transcripts appear equally inhibited by weak and strong age-1 alleles, and one (FIG. 12Y) was suppressed only by the weaker, hx546 allele. The age-1(mg44) mutation up-regulated far fewer genes (13 at P<0.01) than it suppressed (30 at P<0.01), but induced genes include the three with the greatest transcript-level changes (80, 90 and 1000-fold, FIG. 12AG-AI). Among the age-1-stimulated genes, ten were induced exclusively by the mg44 allele (see FIG. 12Z-AD, AH and AI), while the remaining three were induced only 6-12% as much by hx546 as by mg44 (FIG. 12AE-AG).

The 43 genes for which RT-PCR confirmed transcript levels affected chiefly by age-1(mg44) include four ubiquitin-related genes strongly down-regulated by age-1(mg44): two encoding ubiquitin ligases (suppressed 32-fold [FIG. 12A], and 14-fold [FIG. 12I]), ubl-5 (encoding a ubiquitin-like protein; tenfold suppressed [FIG. 12L]), and skr-12 (also encoding a ubiquitin-ligase-complex component; suppressed 3-fold [FIG. 12W]). Other novel, differentially expressed genes, which belong to classes of genes previously implicated in lifespan modulation, include (i) DNA-modifying genes ung-1 (encoding uracil N-glycosylase [FIG. 12J]) and dna-2 (encoding a replication helicase/endonuclease [FIG. 12M]), down-regulated 14- and 10-fold respectively by age-1(mg44); (ii) hsp-12.3, encoding a small heat-shock protein, induced 13-fold [FIG. 12AC]; (iii) dao-4, transcripts of which decline in daf-2-mutant adults at 25° C. [Yu et al., 2001, J Mol Biol 3141017-1028], but are here induced 18-fold [FIG. 12AE] by age-1(mg44); (iv) cyc-2.2, encoding one of two C. elegans cytochrome oxidases, induced 90-fold by age-1(mg44) [FIG. 12AH]; and (v) cyp-35C1, encoding a cytochrome P450, suppressed 17-fold by age-1(mg44) [FIG. 12F].

In addition, many genes displaying age-1-dependent (usually allele-specific) expression were not related in any obvious way to recognized lifespan-modulating pathways. Novel longevity genes down-regulated in age-1 worms include F53H1.3 (suppressed 17-fold by the mg44 allele [FIG. 12G]), encoding an uncharacterized, highly conserved protein; and four genes involved in lipid metabolism: egg-1 encoding an LDL receptor (down 21-fold [FIG. 12C] in mg44), far-2 encoding a fatty-acid and retinol binding protein (repressed 5- and 6-fold by the two age-1 alleles [FIG. 12S]), Ibp-6, encoding another lipid binding protein (suppressed 3-fold by hx546 [FIG. 12Y]), and T21H3.1 predicted to encode a lipase (not shown; suppressed 3.3-fold by mg44). Two VAMP-associated protein genes implicated in phospholipid and inositol metabolism (ZC196.2 and C10H11.7 [FIG. 12AD, AF]) were up-regulated 18- and 21-fold in age-1(mg44) mutant adults; srh-5 and srab-21, encoding serpentine 7-TM receptors, are induced 4.3- and 6-fold [FIG. 12Z, AA]; and Y75B7B.1, encoding a homolog of a cruciform DNA-binding protein, is induced 7-fold [FIG. 12AB] by age-1(mg44). An ATP-dependent RNA helicase gene (R05D11.4 [FIG. 12K]) is down-regulated 13-fold, while nspd-2, encoding an apparent RNA-binding protein, is induced 80-fold [FIG. 12AG] by age-1(mg44). Two C-type lectin genes are pushed in opposite directions, with clec-87 suppressed 16-fold [FIG. 12H] and clec-60 induced 1000-fold [FIG. 12AI] by age-1(mg44).

FIG. 13 presents transcript levels as assessed by RT-PCR for acl-2, a homolog of human AGPAT-alpha, cav-1, a unique worm caveolin gene, tol-1, the worm TLR, gst-5, gst-8, and gst-10 in the different strains. Additionally, FIG. 14 presents the expression profiles of lipid biosynthesis genes, elo-1, elo-2, elo-5, elo-6, fat-1a, fat-1b, fat-4, fat-5a, fat-5b, fat-6, and fat-7, that vary with longevity. FIG. 15 presents the expression profiles of genes that were first identified based on the different protein profiles of long-lived age-1 mutants, which also vary with longevity.

### Example 6

### Functional Testing of Genes Down-Regulated in Age-1 Mutants

F2 age-1(mg44) adults, which are 4- to 5-fold longer-lived than F1 adults, also outperform their parents with respect to resistance to oxidative and electrophilic stresses (See Example 2 and FIG. 3). Relative to N2DRM controls, survival in 5% hydrogen peroxide (taken arbitrarily as time to reach 20% mortality) was extended 2-fold in F1 adults, but 10-fold in their F2 progeny (FIG. 16A). Similarly, 4-HNE survival (time to 20% mortality) increased 1.6-fold in age-1(mg44) F1 worms but 5-fold at F2 worms (FIG. 16B). Although resistance to these stresses returned to almost wild-type levels in double mutants with daf-16, that reversion was not quite complete, whether using the weaker daf-16(m26) allele (not shown) or the daf-16(mu86) deletion allele (FIG. 16A), indicating that such stress-resistance traits were partially mediated by a DAF-16-independent pathway. These results are consistent with apparently incomplete reversion, in daf-16; age-1(mg44) double mutants, of in vitro kinase activity and phosphoprotein levels (FIG. 6) and of transcript levels for several genes (FIG. 10).

To assess the functional importance of transcriptional suppressions associated with extreme longevity, the oxidative-stress resistance of N2DRM wild-type adults exposed to RNAi targeting some of the genes down-regulated in age-1(mg44) F2 adults was measured. After feeding dsRNA-expressing bacteria from the L4 stage onward, day-5 N2DRM adults were tested for survival time in 5 mM H₂O₂. Peroxide resistance paralleled longevity in a series of age-1 mutants (see above) and thus serves as a convenient short-term assay to evaluate the contribution of specific gene-expression changes to the age-1(mg44) phenotype. As summarized in FIG. 16C, RNAi for five of these genes (aka-1, daf-4, aak-1, daf-3, and let-60) significantly enhanced peroxide survival (each P<10⁻³), and a sixth (vps-34) conferred marginal protection (see FIG. 10 legend for proteins encoded). These data establish functional consequences for many of the transcriptional changes observed in F2 age-1(mg44) homozygotes—changes that are likely to contribute to their greatly enhanced stress-resistance and/or longevity.

Transcriptional modulation of the age-1 phenotype, via DAF-16, is hardly surprising in view of substantial evidence that C. elegans IIS is executed predominantly through this transcription factor, by which several hundred targets are regulated either positively or negatively. What was unforeseen was that, in age-1(mg44) F2 worms, essentially the entire IIS pathway would be attenuated at the transcript level. In these very long-lived worms, most of the upstream genes responsible for holding DAF-16 in check are instead silenced by DAF-16 itself, while ins-1 mRNA, encoding an insulin-like peptide that antagonizes the DAF-2 IIS receptor, is 10-fold more abundant than in isogenic controls (see FIGS. 7 and 10). These data imply a positive feedback loop, which amplifies perturbations to create a “flip-flop” with two metastable states. This circuitry, diagrammed in FIG. 16D, normally governs two binary choices: in early larvae, completion of development vs. dauer arrest; and in adults, early reproduction vs. postponed aging and delayed reproduction. In each case, it is essential that the switch be capable of reversal so as to return worms to their reproductive mode when conditions are favorable. F2 worms bearing the age-1(mg44) mutation never regained fertility at 20 or 25° C., and only rarely did so (with 1% frequency) at 15° C.. One interpretation is that IIS signaling in these worms was so severely attenuated as to be irreversibly locked into the pro-longevity, nonreproductive state. In addition to IIS genes, components of many other signaling pathways were also down-regulated exclusively in F2 age-1(mg44) adults. It cannot be inferred that all of these transcriptional effects were beneficial to longevity, but at least 5 of the 11 genes tested contributed significantly and reproducibly to survival of an oxidative stress (FIG. 16C).

Additional genes suppressed in very long-lived age-1(mg44) mutants as assessed by RT-PCR were selected for functional testing and the results are shown in FIG. 16. Of the 21 RNAi constructs targeting genes confirmed by RT-PCR to have decreased expression in age-1 (mg44) worms, 16 (76%) reproducibly conferred significant resistance to 5 mM H₂O₂ (FIG. 17); the RNAi-protective genes were T12E12.1, cpr-1, cyp-35C1, F53H1.3, clec-87, F36F2.3, ung-1, R05D11.4, dna-2, sna-1, far-2, rab-10, R107.2, skr-12, Ibp-6, and twk-18. RNAi knockdown of two genes, Ibp-6 and F52H1.3, was particularly effective in extending lifespan (FIG. 17E).

### Example 7

### Lipid Profiles of Age-1 Mutants

The lipid profiles of N2DRM, various age-1 mutants, and other longevity mutants were analyzed by GC-MS using standard procedures. It was found that age-1(mf44) adults have more short-chain monounsaturated and saturated fatty acids, than do post-gravid wild-type and daf-16; age-1 controls. Fatty acids are shorter in lipids extracted from age-1(nmg44) worms (FIG. 18). Among saturated fatty acids, myristic acid (14:0) is increased and eicosanoic acid (20:0) is decreased in age-1(mg44) worms (FIG. 19). Analysis of the number of double bonds (i.e., double bond index, Pamplona et al, 1998, J Lipid Res 39;1989-1994) revealed that the lipids of age-1(nmg44) worms have significantly fewer double bonds than the lipids from control worms (FIG. 20). Furthermore, longer chain polyunsaturated fatty acids are reduced and shorter chain polyunsaturated fatty acids are increased in age-1(mg44) worms (FIG. 21). These data suggest that the membranes of long-lived worms are altered relative to control worms, and it is predicted that their altered membranes would have a lower susceptibility to lipid peroxidation.

### Example 8

### Phosphatidylinositol Analogs as Inhibitors of PI3K

To determine whether inhibition of PI3K would increase longevity, phosphatidylinositol analogs unable to be phosphorylated at position 3 were administered to wild-type worms and longevity was assayed by measuring survival to the toxin hydrogen peroxide, as detailed above. As shown in FIG. 22, the active phosphatidylinositol ether lipid analogs, PIA24 and PIA7, increased worm survival, while the inactive analog, PIA7 (which lacks an inositol headgroup), had no effect.

What is claimed is:

1. A method for identifying an agent that increases resistance to oxidative and/or electrophilic stress, the method comprising:
a. contacting a test nematode with at least one agent;
b. exposing the test nematode, a negative control nematode, and a mutant nematode to an oxidative and/or electrophilic stress, the mutant nematode being an extremely long-lived mutant nematode that has increased resistance to oxidative and/or electrophilic stress; and
c. comparing the stress responses of the test nematode, the negative control nematode, and the mutant nematode, wherein a change in the response of the test nematode away from the response of the negative control nematode and towards the response of the mutant nematode indicates that the agent provides protection against the oxidative and/or electrophilic stress.

2. The method of claim 1, wherein an agent that increases resistance to the oxidative and/or electrophilic stress also increases lifespan.

3. The method of claim 1, wherein step (a) is performed from about 5 minutes to about 60 minutes before step (b).

4. The method of claim 1, wherein step (a) and step (b) are performed simultaneously.

5. The method of claim 1, wherein the test nematode, the negative control nematode, and the mutant nematode are of the species Caenorhabditis elegans.

6. The method of claim 5, wherein the test nematode and the negative control nematode are substantially identical and are selected from the group consisting of wild type, mutant, and transgenic strains of C. elegans.

7. The method of claim 5, wherein the mutant nematode is at least a second generation mutant selected from the group consisting of an age-1(mg44) null mutant, an age-1(m333) null mutant, and a similar age-1 null or truncation mutant.

8. The method of claim 7, wherein the lifespan of the second generation age-1 null mutant nematode is at least about seven times longer than the lifespan of a wild type nematode.

9. The method of claim 7, wherein the lifespan of the second generation age-1 null mutant nematode is about four times to about five times longer than the lifespan of an age-1 mutant nematode selected from the group consisting of a first generation age-1(mg44) mutant, a first generation age-1(m333) mutant, and an age-1(hx546) mutant.

10. The method of claim 7, wherein an embryo giving rise to the second generation age-1 nematode lacks maternal carryover.

11. The method of claim 10, wherein maternal carryover comprises entities selected from the group consisting of phosphatidylinositol 3-kinase (PI3K), phosphatidylinositol 3,4,5-triphosphate (PIP3), untranslated RNA molecules, and storage proteins.

12. The method of claim 7, wherein the age-1 null mutant nematode develops through the embryonic and larval stages at a temperature below about 25° C..

13. The method of claim 7, wherein the second generation age-1 null mutant nematode has increased resistance to oxidative and/or electrophilic stress relative to a nematode selected from the group consisting of a wild type, a first generation age-1(mg44) mutant, a first generation age-1(m333) mutant, and an age-1(hx546) mutant.

14. The method of claim 7, wherein the test nematode, the negative control nematode, and the mutant nematode are selected from the group consisting of day-1 adults and day-2 adults.

15. The method of claim 7, wherein the test nematode and the negative control nematode are selected from the group consisting of infertile mutant nematodes, day-8 or older wild type postgravid nematode, and day-8 or older moderately long-lived mutant postgravid nematode.

16. The method of claim 15, wherein the moderately long-lived mutant postgravid nematode is selected from the group consisting of an age-1(hx546) homozygous mutant, a daf-2(e1370) homozygous mutant, and a similar moderately long-lived age-1 or daf-2 homozygous mutant.

17. The method of claim 1, wherein exposure to the oxidative and/or electrophilic stress comprises contact with an agent selected from the group consisting of hydrogen peroxide, paraquat, and 4-hydroxynonenal.

18. The method of claim 17, wherein the concentration of hydrogen peroxide ranges from about 1 mm to about 10 mM.

19. The method of claim 17, wherein the concentration of paraquat ranges from about 100 mM to about 200 mM.

20. The method of claim 17, wherein the concentration of 4-hydroxynonenal ranges from about 5 mM to about 20 mM.

21. The method of claim 1, wherein the agent is selected from the group consisting of a small organic molecule, a peptide, a peptidomimetic, an antisense oligonucleotide, an aptomer oligonucleotide, a double stranded RNA interference molecule, and a member of a combinatorial chemical library.

22. The method of claim 1, wherein the agent is a small organic molecule.

23. The method of claim 22, wherein the small organic molecule ranges in size from about 100 Daltons to about 2000 Daltons.

24. The method of claim 1, wherein the agent is selected from the group consisting of a nonphosphorylatable phosphoinositol analog, an inhibitor of PI3K, an inhibitor of PDK-1, and an inhibitor of SGK-1.

25. The method of claim 1, wherein the stress response is assayed in vivo.

26. The method of claim 25, wherein the in vivo assay is short-term survival.

27. The method of claim 26, wherein survival is measured from about 2 hours to about 16 hours after exposure to the oxidative and/or electrophilic stress.

28. The method of claim 1, wherein the stress response is assayed in vitro.

29. The method of claim 28, wherein the in vitro assay is selected from the group consisting of measuring enzyme activity, measuring protein phosphorylation, measuring protein levels, measuring transcript levels, measuring lipid levels, measuring metabolite levels, and analyzing arrays of nucleic acids, proteins, lipids, or small molecules.

30. A method for identifying a biomarker of longevity or a pathway governing longevity in response to phosphatidylinositol 3,4,5-triphosphate (PIP3) signaling, the method comprising:
a. contacting a first nematode with at least one treatment that alters the levels or function of PIP3;
b. exposing the first nematode from step (a) and a second untreated nematode to an oxidative and/or electrophilic stress; and
c. comparing the stress response of the first nematode and the stress response of the second nematode, wherein an increase in resistance to the stress in the first nematode indicates that the treatment altered a longevity biomarker or a PIP3 signaling pathway that mediates longevity.

31. The method of claim 30, wherein the first nematode and the second nematode are of the species Caenorhabditis elegans.

32. The method of claim 31, wherein the first nematode and the second nematode are wild type nematodes.

33. The method of claim 31, wherein the first nematode is an age-1 mutant selected from the group consisting of a first generation age-1(mg44) null mutant, a first generation age-1(m333) null mutant, and an age-1(hx546) mutant, and the second nematode is an extremely long-lived mutant C. elegans nematode.

34. The method of claim 33, wherein the extremely long-lived untreated nematode is an age-1 mutant nematode of at least the second generation selected from the group consisting of an age-1(mg44) null mutant, an age-1(m333) null mutant, and a similar age-1 null or truncation mutant.

35. The method of claim 31, wherein the nematodes are selected from the group consisting of day-1 adults, day-2 adults, and infertile adults.

36. The method of claim 30, wherein a longevity biomarker or a PIP3 signaling pathway governing longevity is used for a therapeutic intervention.

37. The method of claim 30, wherein the treatment is selected from the group consisting of overexpressing a PIP3 interacting protein, blocking expression of a PIP3 interacting protein, introducing a PIP3 interacting protein that has an altered response to PIP3, introducing or overexpressing a pleckstrin homology domain or modification thereof, and introducing or overexpressing a phosphatidylinositol 4,5-diphosphate (PIP2) binding protein, binding domain, or modification thereof.

38. The method of claim 37, wherein the PIP3 interacting protein is selected from the group consisting of PI3 kinase, PDK-1, AKT-11, AKT-2, SGK, SMK-1, and PIP3 phosphatase.

39. The method of claim 37, wherein the PIP3 interacting protein with an altered response to PIP3 is due to a treatment selected from the group consisting of modifying the kinase domain, modifying a phosphorylation site, and modifying the pleckstrin homology domain.

40. The method of claim 30, wherein the treatment is selected from the group consisting of a small organic molecule, a peptide, a peptidomimetic, an antisense oligonucleotide, an aptomer oligonucleotide, a double stranded RNA interference molecule, and a member of a combinatorial chemical library.

41. The method of claim 30, wherein exposure to the oxidative and/or electrophilic stress comprises contact with an agent selected from the group consisting of hydrogen peroxide, paraquat, and 4-hydroxynonenal.

42. The method of claim 30, wherein the stress response is short-term survival.

43. The method of claim 30, wherein step (b) is omitted and step (c) comprises comparing the response of the first nematode and response of the second nematode, wherein the response is selected from the group consisting of lifespan, fertility, fecundity, and developmental timing.

44. A method for identifying a biomarker of longevity or a pathway governing longevity in response to phosphatidylinositol 3,4,5-triphosphate (PIP3) signaling, the method comprising:
a. contacting an extremely long-lived age-1 null mutant C. elegans nematode with at least one constitutively active PIP3 interacting protein;
b. exposing the treated age-1 nematode from step (a) and an untreated extremely long-lived age-1 null mutant nematode to an oxidative and/or electrophilic stress; and
c. comparing the stress responses of the treated age-1 null mutant nematode and the untreated age-1 null mutant nematode, wherein a decrease in resistance to the stress in the treated age-1 null mutant nematode indicates that the protein mediates effects downstream of age-1 to confer the extreme longevity and increased resistance to oxidative and/or electrophilic stress of the extremely long-lived age-1 null mutant nematode.

45. The method of claim 44, wherein a longevity biomarker or a PIP3 signaling pathway governing longevity is used for a therapeutic intervention.

46. The method of claim 44, wherein the treated and untreated age-1 nematodes are homozygous null mutants selected from the group consisting at least a second generation of age-1(mg44) and at least a second generation of age-1(m333).

47. The method of claim 44, wherein the treated and untreated age-1 nematodes are selected from the group consisting of day-1 adults and day-2 adults.

48. The method of claim 44, wherein the PIP3 interacting protein is selected from the group consisting of AKT-1, AKT-2, PDK-1, SGK, and SMK-1.

49. The method of claim 44, wherein the PIP3 interacting protein is made constitutively active by a treatment selected from the group consisting of altering the kinase domain, altering a phosphorylation site, and altering the pleckstrin homology domain.

50. The method of claim 44, wherein exposure to the oxidative and/or electrophilic stress comprises contact with an agent selected from the group consisting of hydrogen peroxide, paraquat, and 4-hydroxynonenal.

51. The method of claim 44, wherein the stress response is short-term survival.

52. The method of claim 44, wherein step (b) is omitted and step (c) comprises comparing the response of the treated age-1 nematode and the response of the untreated age-1 nematode, wherein the response is selected from the group consisting of lifespan, fertility, fecundity, and developmental timing.
